(19) United States
(12) Patent Application Publication
(10) Pub. No.: US 2022/0273433 A1
(43) Pub. Date: Sep. 1, 2022

(54) LEAFLET COAPTATION-ASSISTANCE DEVICES

(71) Applicant: MTEX CARDIO AG, Pfaffikon (CH)

(72) Inventors: Karl-Heinz KUCK, Hamburg (DE); Andrea GUIDOTTI, Zollikon (CH); Pietro GOZZOLI, Zurich (CH)

(21) Appl. No.: 17/632,840

(22) PCT Filed: Aug. 6, 2020
(86) PCT No.: PCT/IB2020/057429

§ 371 Date: Feb. 4, 2022

Related U.S. Application Data
(60) Provisional application No. 62/884,404, filed on Aug. 8, 2019.

(51) Int. Cl. A61F 2/24

(52) U.S. Cl. CPC A61F 2/2454; A61F 2/2418; A61F 2/246; A61F 2220/0008; A61F 2230/0045; A61F 2250/0003

(57) ABSTRACT

A leaflet coaptation-assist device (920, 1020, 1120) is provided for promoting valve coaptation of leaflets (22) of an atrioventricular valve, the leaflet coaptation-assist device (920, 1020, 1120) including a coaptation-assist surface (964, 1064, 1164) and one or more tissue anchors, which are configured to anchor the leaflet coaptation-assist device (920, 1020, 1120) in place within the heart, such that the coaptation-assist surface (964, 1064, 1164) is located at or below the leaflet hinge line and contacts an atrial side of the leaflets (22) during ventricular systole, thereby enhancing leaflet coaptation. In some configurations, the one or more tissue anchors include one or more ventricular loops (988, 1088, 1188), which are configured to couple the leaflet coaptation-assist device (920, 1020, 1120) to the ventricular wall by radial force and friction. Other embodiments are also described.

### CROSS-REFERENCE TO RELATED APPLICATIONS

The present application:

(a) claims priority from U.S. Provisional Patent Application No. 62/884,404, filed Aug. 8, 2019; and

(b) claims priority from and is a continuation-in-part of U.S. patent application Ser. No. 16/529,247, filed Aug. 1, 2019, which is:
(i) a continuation of PCT Application No. PCT/US2018/016811, filed Feb. 5, 2018, which claims priority from U.S. Provisional Patent Application No. 62/455,427, filed Feb. 6, 2017, and U.S. Provisional Patent Application No. 62/570,336, filed Oct. 10, 2017, and
(ii) a continuation of PCT Application No. PCT/IB2018/000145, filed Feb. 5, 2018, which claims priority from U.S. Provisional Patent Application No. 62/455,427, filed Feb. 6, 2017, and U.S. Provisional Patent Application No. 62/570,336, filed Oct. 10, 2017.

All of the above-referenced applications are assigned to the assignee of the present application and incorporated herein by reference.

### FIELD OF THE APPLICATION

The present application relates generally to medical devices and methods. More particularly, this application relates to prosthetic devices and methods for improving the function of prolapsing heart and other circulatory valves.

### BACKGROUND OF THE APPLICATION

Mitral valve insufficiency, either organic (primary) or functional (secondary), such as but not limited to prolapsed, regurgitation, and dithering (MVI), is a valvular heart disease characterized by the displacement of an abnormally thickened mitral valve leaflet into the left atrium during systole which can result in poor coaptation of the individual valve leaflets and valve leakage against backpressure. There are various types of MVI, broadly classified as classic and non-classic. In its non-classic form, MVI carries a low risk of complications and often can be kept minimal by dietary attention. In severe cases of classic MVI, complications include mitral regurgitation, infective endocarditis, congestive heart failure, and, in rare circumstances, cardiac arrest, usually resulting in sudden death. The aortic valve can also suffer from prolapse, and valves of the venous circulation can suffer from a similar condition which can lead to chronic venous insufficiency resulting from damaged or “incompetent” valves which are characterized by poor coaptation.

### SUMMARY OF THE APPLICATION

Embodiments of the present invention provide leaflet coaptation-assist devices and methods for promoting valve coaptation of leaflets of an atrioventricular valve of a heart of a patient. In general, these techniques enhance coaptation by placing a patch or barrier at or near the flexural level (i.e., the leaflet hinge line, demarcated by an annulus) of the atrioventricular valve. Typically, these techniques limit the natural leaflet movement to level at or less than the flexural level. These techniques may be used to improve coaptation of native leaflets or prosthetic leaflets of a prosthetic valve and/or reduce or prevent valve prolapse, thereby minimizing leakage. The techniques described herein may be used for treatment of mitral and tricuspid valve regurgitation and other valvular diseases.

There is therefore provided, in accordance with an Inventive Concept 1 of the present invention, a leaflet coaptation-assist device for promoting valve coaptation of leaflets of an atrioventricular valve of a heart of a patient, the leaflet coaptation-assist device including:

a coaptation-assist surface; and

one or more tissue anchors, which are configured to anchor the leaflet coaptation-assist device in place within the heart of the patient, such that the coaptation-assist surface is located at or below the leaflet hinge line and contacts an atrial side of the leaflets during ventricular systole, thereby enhancing leaflet coaptation, the leaflets selected from the group consisting of: native leaflets and prosthetic leaflets,

wherein the one or more tissue anchors include a non-tissue-piercing anchor, which includes one or more ventricular loops, which are configured to couple the leaflet coaptation-assist device to a ventricular wall by radial force and friction.

Inventive Concept 2. The leaflet coaptation-assist device according to Inventive Concept 1, wherein the one or more ventricular loops are configured to contact one or more of the atrioventricular leaflets at the commissural level and clip the one or more leaflets within the one or more ventricular loops to stabilize anchoring of the leaflet coaptation-assist device.
Inventive Concept 3. The leaflet coaptation-assist device according to Inventive Concept 1, wherein the non-tissue-piercing anchor includes a single ventricular loop.
Inventive Concept 4. The leaflet coaptation-assist device according to Inventive Concept 1, wherein the non-tissue-piercing anchor includes two or more ventricular loops.
Inventive Concept 5. The leaflet coaptation-assist device according to Inventive Concept 4, wherein the two or more ventricular loops or cross one another.
Inventive Concept 6. The leaflet coaptation-assist device according to Inventive Concept 4, wherein the two or more ventricular loops do not cross or touch one another.
Inventive Concept 7. The leaflet coaptation-assist device according to Inventive Concept 1, wherein the one or more ventricular loops are configured to reach to a ventricular apical area.
Inventive Concept 8. The leaflet coaptation-assist device according to Inventive Concept 1, wherein the atrioventricular valve is a mitral valve.
Inventive Concept 9. The leaflet coaptation-assist device according to Inventive Concept 1, wherein the atrioventricular valve is a tricuspid valve.
Inventive Concept 10. The leaflet coaptation-assist device according to Inventive Concept 1, wherein the one or more tissue anchors are configured to anchor the leaflet coaptation-assist device in place within the heart of the patient, such that the coaptation-assist surface is located at the leaflet hinge line and contacts the atrial side of the leaflets during ventricular systole, thereby blocking motion of the leaflets into an atrium and preventing leaflet prolapse.
Inventive Concept 11. The leaflet coaptation-assist device according to Inventive Concept 1, wherein the one or more tissue anchors are configured to anchor the leaflet coaptation-assist device in place within the heart of the patient, such that the coaptation-assist surface is located below the leaflet hinge line such that the leaflets partially coapt with the coaptation-assist surface during ventricular systole.
Inventive Concept 12. The leaflet coaptation-assist device according to any one of Inventive Concepts 1-11, wherein the leaflet coaptation-assist device includes a sheet having a lower surface that provides the coaptation-assist surface.
Inventive Concept 13. The leaflet coaptation-assist device according to Inventive Concept 12, wherein the sheet has four sides.
Inventive Concept 14. The leaflet coaptation-assist device according to Inventive Concept 13, wherein the sheet has a shape of a quadrilateral.
Inventive Concept 15. The leaflet coaptation-assist device according to Inventive Concept 12, wherein the sheet is shaped so as to define three spokes extending radially outward from a central hub region.
Inventive Concept 16. The leaflet coaptation-assist device according to Inventive Concept 15, wherein the three spokes are collectively shaped so as to define a deltoid curve.
Inventive Concept 17. The leaflet coaptation-assist device according to Inventive Concept 13, wherein the sheet has two straight sides and two curved sides.
Inventive Concept 18. The leaflet coaptation-assist device according to Inventive Concept 17, wherein the sheet has two straight shorter sides and two curved longer sides.
Inventive Concept 19. The leaflet coaptation-assist device according to Inventive Concept 17, wherein the sheet has two straight longer sides and two curved shorter sides.
Inventive Concept 20. The leaflet coaptation-assist device according to Inventive Concept 12, wherein the sheet has two longer sides and two shorter sides, and the shorter sides include respective stiffened portions.
Inventive Concept 21. The leaflet coaptation-assist device according to any one of Inventive Concepts 1-11, wherein the leaflet coaptation-assist device includes a thick structure having a lower surface that provides the coaptation-assist surface.
Inventive Concept 22. The leaflet coaptation-assist device according to Inventive Concept 21, wherein the thick structure includes a solid foam.
Inventive Concept 23. The leaflet coaptation-assist device according to Inventive Concept 21, wherein the thick structure includes at least one balloon.
Inventive Concept 24. The leaflet coaptation-assist device according to Inventive Concept 21, wherein the thick structure is curved on a ventricular side thereof.
Inventive Concept 25. The leaflet coaptation-assist device according to Inventive Concept 24, wherein the thick structure is multi-curved on the ventricular side thereof.
Inventive Concept 26. The leaflet coaptation-assist device according to Inventive Concept 21, wherein the thick structure is flat on an atrial side thereof.
Inventive Concept 27. The leaflet coaptation-assist device according to Inventive Concept 21, wherein the thick structure has a greatest thickness in the atrial-ventricular direction of at least 3 mm.
Inventive Concept 28. The leaflet coaptation-assist device according to Inventive Concept 27, wherein the thick structure has a greatest thickness in the atrial-ventricular direction of no more than 40 mm.

There is further provided, in accordance with an Inventive Concept 29 of the present invention, a leaflet coaptation-assist device for promoting valve coaptation of leaflets of a mitral valve of a heart of a patient, the leaflet coaptation-assist device including:

a sheet having a lower surface that defines a coaptation-assist surface, and two longer sides and two shorter sides, the shorter sides including respective stiffened portions; and

a plurality of tissue-piercing tissue anchors, which are configured to be coupled to a structure selected from the group consisting of: an annulus of the mitral valve and an annuloplasty ring implanted on the annulus of the mitral valve, so as to anchor the leaflet coaptation-assist device in place within the heart of the patient, such that the coaptation-assist surface is located at or below the leaflet hinge line and contacts an atrial side of the leaflets during systole, thereby enhancing leaflet coaptation, the leaflets selected from the group consisting of: native leaflets and prosthetic leaflets.

Inventive Concept 30. The leaflet coaptation-assist device according to Inventive Concept 29, wherein the tissue anchors are configured to anchor the leaflet coaptation-assist device in place within the heart of the patient, such that the coaptation-assist surface is located at the leaflet hinge line and contacts the atrial side of the leaflets during systole, thereby blocking motion of the leaflets into an atrium and preventing leaflet prolapse.
Inventive Concept 31. The leaflet coaptation-assist device according to Inventive Concept 29, wherein the tissue anchors are configured to anchor the leaflet coaptation-assist device in place within the heart of the patient, such that the coaptation-assist surface is located below the leaflet hinge line such that the leaflets partially coapt with the coaptation-assist surface during systole.
Inventive Concept 32. The leaflet coaptation-assist device according to Inventive Concept 29, wherein each of the stiffened portions is formed by two layers of the sheet or by a folding of the sheet.
Inventive Concept 33. The leaflet coaptation-assist device according to Inventive Concept 29, wherein the stiffened portions include respective stiffener elements.
Inventive Concept 34. The leaflet coaptation-assist device according to Inventive Concept 29, wherein the tissue anchors are configured to anchor the leaflet coaptation-assist device to an annulus of the mitral valve.
Inventive Concept 35. The leaflet coaptation-assist device according to Inventive Concept 29, wherein the tissue anchors are configured to anchor the leaflet coaptation-assist device to an annuloplasty ring implanted on an annulus of the mitral valve.
Inventive Concept 36. The leaflet coaptation-assist device according to Inventive Concept 29, wherein the sheet has exactly four sides.
Inventive Concept 37. The leaflet coaptation-assist device according to Inventive Concept 36, wherein the sheet has a shape of a quadrilateral.
Inventive Concept 38. The leaflet coaptation-assist device according to Inventive Concept 36, wherein the sheet has two straight sides and two curved sides.
Inventive Concept 39. The leaflet coaptation-assist device according to Inventive Concept 38, wherein the two shorter sides are straight and the two longer sides are curved.
Inventive Concept 40. The leaflet coaptation-assist device according to Inventive Concept 38, wherein the two longer sides are straight and the shorter sides are curved.
Inventive Concept 41. The leaflet coaptation-assist device according to Inventive Concept 29, wherein the tissue-piercing anchors include respective helical tissue-piercing coupling-elements.
Inventive Concept 42. The leaflet coaptation-assist device according to any one of Inventive Concepts 29-41, wherein the lower surface of the sheet has an elliptically convex curvature.
Inventive Concept 43. The leaflet coaptation-assist device according to Inventive Concept 42, further including a curved stiffener that is coupled to the sheet and provides the elliptically convex curvature.
Inventive Concept 44. The leaflet coaptation-assist device according to any one of Inventive Concepts 29-41, wherein the lower surface of the sheet has a bi-lobal convex curvature having two rounded curvature shapes.
Inventive Concept 45. The leaflet coaptation-assist device according to Inventive Concept 44, further including one or more curved stiffeners that are coupled to the sheet and provide the bi-lobal convex curvature.
Inventive Concept 46. The leaflet coaptation-assist device according to Inventive Concept 43, wherein the curved stiffener includes a material having a shape memory.
Inventive Concept 47. The leaflet coaptation-assist device according to any one of Inventive Concepts 29-41, wherein the tissue-piercing anchors are fixed to the coaptation-assist surface along the two shorter sides of the sheet.
Inventive Concept 48. The leaflet coaptation-assist device according to Inventive Concept 47, wherein the tissue-piercing anchors are fixed to the coaptation-assist surface at or near respective centers of the two shorter sides of the sheet.
Inventive Concept 49. The leaflet coaptation-assist device according to Inventive Concept 47, wherein the tissue-piercing anchors are fixed to the coaptation-assist surface at or near respective ends of the two shorter sides of the sheet.
Inventive Concept 50. The leaflet coaptation-assist device according to any one of Inventive Concepts 29-41, wherein the tissue-piercing anchors are separate and discrete from the leaflet coaptation-assist device, and are configured to be coupled to the leaflet coaptation-assist device and the structure in situ during an implantation procedure.

There is still further provided, in accordance with an Inventive Concept 51 of the present invention, a leaflet coaptation-assist device for promoting valve coaptation of leaflets of a mitral valve of a heart of a patient, the leaflet coaptation-assist device including:

a sheet having two longer sides and two shorter sides, and a lower surface that defines a coaptation-assist surface and has a convex curvature that is (a) elliptic or (b) bi-lobal having two rounded curvature shapes; and

one or more tissue anchors, which are configured to anchor the leaflet coaptation-assist device in place within the heart of the patient, such that the coaptation-assist surface is located at or below the leaflet hinge line and contacts an atrial side of the leaflets during systole, thereby enhancing leaflet coaptation, the leaflets selected from the group consisting of: native leaflets and prosthetic leaflets.

Inventive Concept 52. The leaflet coaptation-assist device according to Inventive Concept 51, wherein the one or more tissue anchors are configured to anchor the leaflet coaptation-assist device in place within the heart of the patient, such that the coaptation-assist surface is located at the leaflet hinge line and contacts the atrial side of the leaflets during systole, thereby blocking motion of the leaflets into an atrium and preventing leaflet prolapse.
Inventive Concept 53. The leaflet coaptation-assist device according to Inventive Concept 51, wherein the one or more tissue anchors are configured to anchor the leaflet coaptation-assist device in place within the heart of the patient, such that the coaptation-assist surface is located below the leaflet hinge line such that the leaflets partially coapt with the coaptation-assist surface during systole.
Inventive Concept 54. The leaflet coaptation-assist device according to Inventive Concept 51, wherein the convex curvature is elliptic.
Inventive Concept 55. The leaflet coaptation-assist device according to Inventive Concept 51, wherein the convex curvature is bi-lobal.
Inventive Concept 56. The leaflet coaptation-assist device according to Inventive Concept 51, wherein the one or more tissue anchors are configured to anchor the leaflet coaptation-assist device to an annulus of the mitral valve.
Inventive Concept 57. The leaflet coaptation-assist device according to Inventive Concept 51, wherein the one or more tissue anchors are configured to anchor the leaflet coaptation-assist device to an annuloplasty ring implanted on an annulus of the mitral valve.
Inventive Concept 58. The leaflet coaptation-assist device according to any one of Inventive Concepts 51-57, further including a curved stiffener that is coupled to the sheet and provides the convex curvature.
Inventive Concept 59. The leaflet coaptation-assist device according to Inventive Concept 58, wherein the curved stiffener includes a material having a shape memory.
Inventive Concept 60. The leaflet coaptation-assist device according to any one of Inventive Concepts 51-57, wherein the shorter sides include respective stiffened portions.
Inventive Concept 61. The leaflet coaptation-assist device according to Inventive Concept 60, wherein each of the stiffened portions is formed by two layers of the sheet or by a folding of the sheet.
Inventive Concept 62. The leaflet coaptation-assist device according to Inventive Concept 60, wherein the stiffened portions include respective stiffener elements.
Inventive Concept 63. The leaflet coaptation-assist device according to any one of Inventive Concepts 51-57, wherein the sheet has exactly four sides.
Inventive Concept 64. The leaflet coaptation-assist device according to Inventive Concept 63, wherein the sheet has a shape of a quadrilateral.
Inventive Concept 65. The leaflet coaptation-assist device according to Inventive Concept 63, wherein the sheet has two straight sides and two curved sides.
Inventive Concept 66. The leaflet coaptation-assist device according to Inventive Concept 65, wherein the two shorter sides are straight and the two longer sides are curved.
Inventive Concept 67. The leaflet coaptation-assist device according to Inventive Concept 65, wherein the two longer sides are straight and the shorter sides are curved.
Inventive Concept 68. The leaflet coaptation-assist device according to any one of Inventive Concepts 51-57, wherein the one or more tissue anchors include a plurality of tissue-piercing anchors, which are configured to be coupled to a structure selected from the group consisting of: an annulus of the mitral valve and an annuloplasty ring implanted on the annulus of the mitral valve.
Inventive Concept 69. The leaflet coaptation-assist device according to Inventive Concept 68, wherein the tissue-piercing anchors include respective helical tissue-piercing coupling-elements.
Inventive Concept 70. The leaflet coaptation-assist device according to Inventive Concept 68, wherein the tissue-piercing anchors are fixed to the coaptation-assist surface along the two shorter sides of the sheet.
Inventive Concept 71. The leaflet coaptation-assist device according to Inventive Concept 70, wherein the tissue-piercing anchors are fixed to the coaptation-assist surface at or near respective centers of the two shorter sides of the sheet.
Inventive Concept 72. The leaflet coaptation-assist device according to Inventive Concept 70, wherein the tissue-piercing anchors are fixed to the coaptation-assist surface at or near respective ends of the two shorter sides of the sheet.
Inventive Concept 73. The leaflet coaptation-assist device according to Inventive Concept 68, wherein the tissue-piercing anchors are separate and discrete from the leaflet coaptation-assist device, and are configured to be coupled to the leaflet coaptation-assist device and the structure in situ during an implantation procedure.

There is additionally provided, in accordance with an Inventive Concept 74 of the present invention, a leaflet coaptation-assist device for promoting valve coaptation of leaflets of an atrioventricular valve of a heart of a patient, the leaflet coaptation-assist device including:

a thick structure having a lower surface that provides a coaptation-assist surface, wherein the thick structure is curved on a ventricular side thereof; and

one or more tissue anchors, which are configured to anchor the leaflet coaptation-assist device in place within the heart of the patient, such that the coaptation-assist surface is located at or below the leaflet hinge line and contacts an atrial side of the leaflets during ventricular systole, thereby enhancing leaflet coaptation, the leaflets selected from the group consisting of: native leaflets and prosthetic leaflets.

Inventive Concept 75. The leaflet coaptation-assist device according to Inventive Concept 74, wherein the thick structure includes a solid foam.
Inventive Concept 76. The leaflet coaptation-assist device according to Inventive Concept 74, wherein the thick structure includes at least one balloon.
Inventive Concept 77. The leaflet coaptation-assist device according to Inventive Concept 74, wherein the atrioventricular valve is a mitral valve.
Inventive Concept 78. The leaflet coaptation-assist device according to Inventive Concept 74, wherein the atrioventricular valve is a tricuspid valve.
Inventive Concept 79. The leaflet coaptation-assist device according to Inventive Concept 74, wherein the one or more tissue anchors are configured to anchor the leaflet coaptation-assist device in place within the heart of the patient, such that the coaptation-assist surface is located at the leaflet hinge line and contacts the atrial side of the leaflets during ventricular systole, thereby blocking motion of the leaflets into an atrium and preventing leaflet prolapse.
Inventive Concept 80. The leaflet coaptation-assist device according to Inventive Concept 74, wherein the one or more tissue anchors are configured to anchor the leaflet coaptation-assist device in place within the heart of the patient, such that the coaptation-assist surface is located below the leaflet hinge line such that the leaflets partially coapt with the coaptation-assist surface during ventricular systole.
Inventive Concept 81. The leaflet coaptation-assist device according to Inventive Concept 74, wherein the one or more tissue anchors are configured to anchor the leaflet coaptation-assist device to an annulus of the atrioventricular valve.
Inventive Concept 82. The leaflet coaptation-assist device according to Inventive Concept 74, wherein the one or more tissue anchors are configured to anchor the leaflet coaptation-assist device to an annuloplasty ring implanted on an annulus of the atrioventricular valve.
Inventive Concept 83. The leaflet coaptation-assist device according to Inventive Concept 74, wherein the one or more tissue anchors include a plurality of tissue-piercing anchors, which are configured to be coupled to a structure selected from the group consisting of: an annulus of the atrioventricular valve and an annuloplasty ring implanted on the annulus of the atrioventricular valve.
Inventive Concept 84. The leaflet coaptation-assist device according to Inventive Concept 83, wherein the tissue-piercing anchors include respective helical tissue-piercing coupling-elements.
Inventive Concept 85. The leaflet coaptation-assist device according to Inventive Concept 83, wherein the coaptation-assist surface has four sides, and wherein the tissue-piercing anchors are fixed to the coaptation-assist surface along two shorter sides of the coaptation-assist surface.
Inventive Concept 86. The leaflet coaptation-assist device according to Inventive Concept 85, wherein the tissue-piercing anchors are fixed to the coaptation-assist surface at or near respective centers of the two shorter sides of the coaptation-assist surface.
Inventive Concept 87. The leaflet coaptation-assist device according to Inventive Concept 85, wherein the tissue-piercing anchors are fixed to the coaptation-assist surface at or near respective ends of the two shorter sides of the coaptation-assist surface.
Inventive Concept 88. The leaflet coaptation-assist device according to Inventive Concept 83, wherein the tissue-piercing anchors are separate and discrete from the leaflet coaptation-assist device, and are configured to be coupled to the leaflet coaptation-assist device and the structure in situ during an implantation procedure.
Inventive Concept 89. The leaflet coaptation-assist device according to Inventive Concept 74, wherein the one or more tissue anchors include a non-tissue-piercing anchor, which is configured to couple the leaflet coaptation-assist device to a native cardiac structure.
Inventive Concept 90. The leaflet coaptation-assist device according to Inventive Concept 89, wherein the non-tissue-piercing anchor includes a stent, which is configured to couple the leaflet coaptation-assist device to atrial tissue by radial force and friction.
Inventive Concept 91. The leaflet coaptation-assist device according to Inventive Concept 89, wherein the non-tissue-piercing anchor includes one or more ventricular loops, which are configured to couple the leaflet coaptation-assist device to a ventricular wall by radial force and friction.
Inventive Concept 92. The leaflet coaptation-assist device according to Inventive Concept 91, wherein the one or more ventricular loops are configured to contact one or more of the atrioventricular leaflets at the commissural level and clip the one or more leaflets within the one or more ventricular loops to stabilize anchoring of the leaflet coaptation-assist device.
Inventive Concept 93. The leaflet coaptation-assist device according to Inventive Concept 74, wherein the thick structure has a greatest thickness in the atrial-ventricular direction of at least 3 mm.
Inventive Concept 94. The leaflet coaptation-assist device according to Inventive Concept 93, wherein the thick structure has a greatest thickness in the atrial-ventricular direction of no more than 40 mm.
Inventive Concept 95. The leaflet coaptation-assist device according to any one of Inventive Concepts 74-94, wherein the thick structure is multi-curved on the ventricular side thereof.
Inventive Concept 96. The leaflet coaptation-assist device according to any one of Inventive Concepts 74-94, wherein the thick structure is flat on an atrial side thereof.

There is yet additionally provided, in accordance with an Inventive Concept 97 of the present invention, a leaflet coaptation-assist device for promoting valve coaptation of leaflets of a tricuspid valve of a heart of a patient, the leaflet coaptation-assist device including:

a sheet, which is shaped so as to define three spokes extending radially outward from a central hub region, and which as a lower surface that defines a coaptation-assist surface; and

one or more tissue anchors, which are configured to anchor the leaflet coaptation-assist device in place within the heart of the patient, such that the coaptation-assist surface is located at or below the leaflet hinge line and contacts an atrial side of the leaflets during ventricular systole, thereby enhancing leaflet coaptation, the leaflets selected from the group consisting of: native leaflets and prosthetic leaflets.

Inventive Concept 98. The leaflet coaptation-assist device according to Inventive Concept 97, wherein the three spokes are collectively shaped so as to define a deltoid curve.
Inventive Concept 99. The leaflet coaptation-assist device according to Inventive Concept 97, wherein radially-outward ends of the spokes include respective stiffened portions.
Inventive Concept 100. The leaflet coaptation-assist device according to any one of Inventive Concepts 97-99, wherein the one or more tissue anchors include a non-tissue-piercing anchor, which includes one or more ventricular loops, which extend in a ventricular direction from the sheet to stabilize the leaflet coaptation-assist device within a ventricular chamber.
Inventive Concept 101. The leaflet coaptation-assist device according to any one of Inventive Concepts 97-99, wherein the one or more tissue anchors include a plurality of tissue-piercing anchors.

There is also provided, in accordance with an Inventive Concept 102 of the present invention, a leaflet coaptation-assist device for promoting valve coaptation of leaflets of a mitral valve of a heart of a patient, the leaflet coaptation-assist device including:

a sheet having two longer sides and two shorter sides, and a lower surface that defines a coaptation-assist surface; and

one or more tissue anchors, which are configured to anchor the leaflet coaptation-assist device in place within the heart of the patient, such that the two shorter sides are disposed along two respective commissural areas of an annulus of the mitral valve near respective commissures of the mitral valve, and the coaptation-assist surface is located at or below the leaflet hinge line and contacts respective atrial sides of the leaflets during systole, such that both of the leaflets coapt with the coaptation-assist surface during systole, the leaflets selected from the group consisting of: native leaflets and prosthetic leaflets.

Inventive Concept 103. The leaflet coaptation-assist device according to Inventive Concept 102, wherein the leaflet coaptation-assist device is configured such that when the leaflet coaptation-assist device is anchored in place within the heart, during systole, respective free edges of the leaflets coapt with the coaptation-assist surface along entire lengths of the free edges, such that the coaptation-assist surface entirely covers an orifice of the mitral valve.
Inventive Concept 104. The leaflet coaptation-assist device according to Inventive Concept 102, wherein the one or more tissue anchors are configured to anchor the leaflet coaptation-assist device in place within the heart of the patient, such that the coaptation-assist surface is located at the leaflet hinge line and contacts the atrial sides of the leaflets during systole, thereby blocking motion of the leaflets into an atrium and preventing leaflet prolapse.
Inventive Concept 105. The leaflet coaptation-assist device according to Inventive Concept 102, wherein the one or more tissue anchors are configured to anchor the leaflet coaptation-assist device in place within the heart of the patient, such that the coaptation-assist surface is located below the leaflet hinge line such that the leaflets partially coapt with the coaptation-assist surface during systole.
Inventive Concept 106. The leaflet coaptation-assist device according to Inventive Concept 102, wherein the one or more tissue anchors are configured to anchor the leaflet coaptation-assist device to the annulus of the mitral valve.
Inventive Concept 107. The leaflet coaptation-assist device according to Inventive Concept 102, wherein the one or more tissue anchors are configured to anchor the leaflet coaptation-assist device to an annuloplasty ring implanted on the annulus of the mitral valve.
Inventive Concept 108. The leaflet coaptation-assist device according to Inventive Concept 102, wherein the sheet has exactly four sides.
Inventive Concept 109. The leaflet coaptation-assist device according to Inventive Concept 108, wherein the sheet has a shape of a quadrilateral.
Inventive Concept 110. The leaflet coaptation-assist device according to Inventive Concept 102, wherein the sheet has two straight sides and two curved sides.
Inventive Concept 111. The leaflet coaptation-assist device according to Inventive Concept 110, wherein the two shorter sides of the sheet are straight and the two longer sides of the sheet are curved.
Inventive Concept 112. The leaflet coaptation-assist device according to Inventive Concept 110, wherein the two longer sides of the sheet are straight and the two shorter sides of the sheet are curved.
Inventive Concept 113. The leaflet coaptation-assist device according to Inventive Concept 102, wherein the shorter sides include respective stiffened portions.
Inventive Concept 114. The leaflet coaptation-assist device according to any one of Inventive Concepts 102-113, wherein the one or more tissue anchors include a plurality of tissue-piercing anchors, which are configured to be coupled to a structure selected from the group consisting of: the annulus of the mitral valve and an annuloplasty ring implanted on the annulus of the mitral valve.
Inventive Concept 115. The leaflet coaptation-assist device according to Inventive Concept 114, wherein the tissue-piercing anchors include respective helical tissue-piercing coupling-elements.
Inventive Concept 116. The leaflet coaptation-assist device according to Inventive Concept 114, wherein the tissue-piercing anchors are fixed to the coaptation-assist surface along the two shorter sides of the sheet.
Inventive Concept 117. The leaflet coaptation-assist device according to Inventive Concept 116, wherein the tissue-piercing anchors are fixed to the coaptation-assist surface at or near respective centers of the two shorter sides of the sheet.
Inventive Concept 118. The leaflet coaptation-assist device according to Inventive Concept 116, wherein the tissue-piercing anchors are fixed to the coaptation-assist surface at or near respective ends of the two shorter sides of the sheet.
Inventive Concept 119. The leaflet coaptation-assist device according to Inventive Concept 114, wherein the tissue-piercing anchors are separate and discrete from the leaflet coaptation-assist device, and are configured to be coupled to the leaflet coaptation-assist device and the structure in situ during an implantation procedure.
Inventive Concept 120. The leaflet coaptation-assist device according to any one of Inventive Concepts 102-113, wherein the one or more tissue anchors include a non-tissue-piercing anchor, which is configured to couple the leaflet coaptation-assist device to a native cardiac structure.
Inventive Concept 121. The leaflet coaptation-assist device according to Inventive Concept 120, wherein the non-tissue-piercing anchor includes a stent, which is configured to couple the leaflet coaptation-assist device to atrial tissue by radial force and friction.
Inventive Concept 122. The leaflet coaptation-assist device according to Inventive Concept 120, wherein the non-tissue-piercing anchor includes one or more ventricular loops, which are configured to couple the leaflet coaptation-assist device to a ventricular wall by radial force and friction.
Inventive Concept 123. The leaflet coaptation-assist device according to Inventive Concept 122, wherein the one or more ventricular loops are configured to contact one or more of the leaflets at the commissural level and clip the one or more leaflets within the one or more ventricular loops to stabilize anchoring of the leaflet coaptation-assist device.
Inventive Concept 124. The leaflet coaptation-assist device according to Inventive Concept 122, wherein the non-tissue-piercing anchor includes a single ventricular loop.
Inventive Concept 125. The leaflet coaptation-assist device according to Inventive Concept 122, wherein the non-tissue-piercing anchor includes two or more ventricular loops.
Inventive Concept 126. The leaflet coaptation-assist device according to Inventive Concept 125, wherein the two or more ventricular loops or cross one another.
Inventive Concept 127. The leaflet coaptation-assist device according to Inventive Concept 125, wherein the two or more ventricular loops do not cross or touch one another.
Inventive Concept 128. The leaflet coaptation-assist device according to Inventive Concept 122, wherein the one or more ventricular loops are configured to reach to a ventricular apical area.

There is further provided, in accordance with an Inventive Concept 129 of the present invention, a leaflet coaptation-assist device for promoting valve coaptation of leaflets of a mitral valve of a heart of a patient, the leaflet coaptation-assist device including:

a coaptation-assist surface having a convex curvature; and

one or more tissue anchors, which are configured to anchor the leaflet coaptation-assist device in place within the heart of the patient, such that the coaptation-assist surface is located at or below the leaflet hinge line and contacts an atrial side of the leaflets during systole, thereby enhancing leaflet coaptation, the leaflets selected from the group consisting of: native leaflets and prosthetic leaflets.

Inventive Concept 130. The leaflet coaptation-assist device according to Inventive Concept 129, wherein the one or more tissue anchors are configured to anchor the leaflet coaptation-assist device in place within the heart of the patient, such that the coaptation-assist surface is located at the leaflet hinge line and contacts the atrial side of the leaflets during systole, thereby blocking motion of the leaflets into an atrium and preventing leaflet prolapse.
Inventive Concept 131. The leaflet coaptation-assist device according to Inventive Concept 129, wherein the one or more tissue anchors are configured to anchor the leaflet coaptation-assist device in place within the heart of the patient, such that the coaptation-assist surface is located below the leaflet hinge line such that the leaflets partially coapt with the coaptation-assist surface during systole.
Inventive Concept 132. The leaflet coaptation-assist device according to Inventive Concept 129, wherein the one or more tissue anchors are configured to anchor the leaflet coaptation-assist device to an annulus of the mitral valve.
Inventive Concept 133. The leaflet coaptation-assist device according to Inventive Concept 129, wherein the one or more tissue anchors are configured to anchor the leaflet coaptation-assist device to an annuloplasty ring implanted on an annulus of the mitral valve.
Inventive Concept 134. The leaflet coaptation-assist device according to Inventive Concept 129, wherein the convex curvature is elliptic.
Inventive Concept 135. The leaflet coaptation-assist device according to Inventive Concept 129, wherein the convex curvature is bi-lobal having two rounded curvature shapes.
Inventive Concept 136. The leaflet coaptation-assist device according to any one of Inventive Concepts 129-135, wherein the leaflet coaptation-assist device includes a sheet having two longer sides and two shorter sides, and a lower surface that defines the coaptation-assist surface.
Inventive Concept 137. The leaflet coaptation-assist device according to Inventive Concept 136, further including a curved stiffener that is coupled to the sheet and provides the convex curvature.
Inventive Concept 138. The leaflet coaptation-assist device according to Inventive Concept 137, wherein the curved stiffener includes a material having a shape memory.
Inventive Concept 139. The leaflet coaptation-assist device according to Inventive Concept 136, wherein the shorter sides include respective stiffened portions.
Inventive Concept 140. The leaflet coaptation-assist device according to Inventive Concept 139, wherein each of the stiffened portions is formed by two layers of the sheet or by a folding of the sheet.
Inventive Concept 141. The leaflet coaptation-assist device according to Inventive Concept 139, wherein the stiffened portions include respective stiffener elements.
Inventive Concept 142. The leaflet coaptation-assist device according to Inventive Concept 136, wherein the sheet has exactly four sides.
Inventive Concept 143. The leaflet coaptation-assist device according to Inventive Concept 142, wherein the sheet has a shape of a quadrilateral.
Inventive Concept 144. The leaflet coaptation-assist device according to Inventive Concept 142, wherein the sheet has two straight sides and two curved sides.
Inventive Concept 145. The leaflet coaptation-assist device according to Inventive Concept 144, wherein the two shorter sides are straight and the two longer sides are curved.
Inventive Concept 146. The leaflet coaptation-assist device according to Inventive Concept 144, wherein the two longer sides are straight and the shorter sides are curved.
Inventive Concept 147. The leaflet coaptation-assist device according to Inventive Concept 136, wherein the one or more tissue anchors include a plurality of tissue-piercing anchors, which are configured to be coupled to a structure selected from the group consisting of: an annulus of the mitral valve and an annuloplasty ring implanted on the annulus of the mitral valve.
Inventive Concept 148. The leaflet coaptation-assist device according to Inventive Concept 147, wherein the tissue-piercing anchors include respective helical tissue-piercing coupling-elements.
Inventive Concept 149. The leaflet coaptation-assist device according to Inventive Concept 147, wherein the tissue-piercing anchors are fixed to the coaptation-assist surface along the two shorter sides of the sheet.
Inventive Concept 150. The leaflet coaptation-assist device according to Inventive Concept 149, wherein the tissue-piercing anchors are fixed to the coaptation-assist surface at or near respective centers of the two shorter sides of the sheet.
Inventive Concept 151. The leaflet coaptation-assist device according to Inventive Concept 149, wherein the tissue-piercing anchors are fixed to the coaptation-assist surface at or near respective ends of the two shorter sides of the sheet.
Inventive Concept 152. The leaflet coaptation-assist device according to any one of Inventive Concepts 129-135, wherein the one or more tissue anchors include a plurality of tissue-piercing anchors, which are configured to be coupled to a structure selected from the group consisting of: an annulus of the mitral valve and an annuloplasty ring implanted on the annulus of the mitral valve.
Inventive Concept 153. The leaflet coaptation-assist device according to Inventive Concept 152, wherein the tissue-piercing anchors are separate and discrete from the leaflet coaptation-assist device, and are configured to be coupled to the leaflet coaptation-assist device and the structure in situ during an implantation procedure.

There is further provided, in accordance with an Inventive Concept 154 of the present invention, a method for promoting valve coaptation in a patient, the method including:

identifying incomplete coaptation of leaflets of a mitral valve of the patient, the leaflets selected from the group consisting of: native leaflets and prosthetic leaflets; and

implanting a leaflet coaptation-assist device that includes a sheet having two longer sides and two shorter sides, such that the two shorter sides are disposed along two respective commissural areas of an annulus of the mitral valve near respective commissures of the mitral valve, and a coaptation-assist surface defined by a lower surface of the sheet is located at or below the leaflet hinge line and contacts respective atrial sides of the leaflets during systole, such that both of the leaflets coapt with the coaptation-assist surface during systole.

Inventive Concept 155. The method according to Inventive Concept 154, wherein implanting the leaflet coaptation-assist device includes implanting the leaflet coaptation-assist device such that, during systole, respective free edges of the leaflets coapt with the coaptation-assist surface along entire lengths of the free edges, such that the coaptation-assist surface entirely covers an orifice of the mitral valve.
Inventive Concept 156. The method according to Inventive Concept 154, wherein implanting the leaflet coaptation-assist device includes implanting the leaflet coaptation-assist device such that, during systole, (a) a first free edge of a first one of the leaflets coapts with the coaptation-assist surface along a majority of a first length of the first free edge, and (b) a second free edge of a second one of the leaflets coapts with the coaptation-assist surface along a majority of a second length of the second free edge.
Inventive Concept 157. The method according to Inventive Concept 156, wherein implanting the leaflet coaptation-assist device includes implanting the leaflet coaptation-assist device such that, during systole, the first free edge of the first one of the leaflets coapts with the coaptation-assist surface along an entirety of the first length of the first free edge.
Inventive Concept 158. The method according to Inventive Concept 154, wherein implanting the leaflet coaptation-assist device includes implanting leaflet coaptation-assist device such that the coaptation-assist surface is located at the leaflet hinge line and contacts the atrial sides of the leaflets during systole, thereby blocking motion of the leaflets into an atrium and preventing leaflet prolapse.
Inventive Concept 159. The method according to Inventive Concept 154, wherein implanting the leaflet coaptation-assist device includes implanting leaflet coaptation-assist device such that the coaptation-assist surface is located below the leaflet hinge line such that the leaflets partially coapt with the coaptation-assist surface during systole.
Inventive Concept 160. The method according to Inventive Concept 154, wherein implanting the leaflet coaptation-assist device includes coupling the leaflet coaptation-assist device to the annulus of the mitral valve.
Inventive Concept 161. The method according to Inventive Concept 154, wherein implanting the leaflet coaptation-assist device includes coupling the leaflet coaptation-assist device to an annuloplasty ring implanted on the annulus of the mitral valve.
Inventive Concept 162. The method according to Inventive Concept 154, wherein the sheet has exactly four sides.
Inventive Concept 163. The method according to Inventive Concept 162, wherein the sheet has a shape of a quadrilateral.
Inventive Concept 164. The method according to Inventive Concept 154, wherein the sheet has two straight sides and two curved sides.
Inventive Concept 165. The method according to Inventive Concept 164, wherein the two shorter sides of the sheet are straight and the two longer sides of the sheet are curved.
Inventive Concept 166. The method according to Inventive Concept 164, wherein the two longer sides of the sheet are straight and the two shorter sides of the sheet are curved.
Inventive Concept 167. The method according to Inventive Concept 154, wherein the shorter sides include respective stiffened portions.
Inventive Concept 168. The method according to Inventive Concept 154, wherein the leaflet coaptation-assist device includes a plurality of tissue-piercing anchors, and wherein implanting the leaflet coaptation-assist device includes coupling the tissue-piercing anchors to a structure selected from the group consisting of: the annulus of the mitral valve and an annuloplasty ring implanted on the annulus of the mitral valve.
Inventive Concept 169. The method according to Inventive Concept 168, wherein the tissue-piercing anchors include respective helical tissue-piercing coupling-elements.
Inventive Concept 170. The method according to Inventive Concept 168, wherein the tissue-piercing anchors are fixed to the coaptation-assist surface along the two shorter sides of the sheet.
Inventive Concept 171. The method according to Inventive Concept 170, wherein the tissue-piercing anchors are fixed to the coaptation-assist surface at or near respective centers of the two shorter sides of the sheet.
Inventive Concept 172. The method according to Inventive Concept 170, wherein the tissue-piercing anchors are fixed to the coaptation-assist surface at or near respective ends of the two shorter sides of the sheet.
Inventive Concept 173. The method according to Inventive Concept 168, wherein the tissue-piercing anchors are separate and discrete from the leaflet coaptation-assist device, and wherein implanting the leaflet coaptation-assist device includes coupling the tissue-piercing anchors to the leaflet coaptation-assist device and the structure in situ during an implantation procedure.
Inventive Concept 174. The method according to Inventive Concept 154,

wherein the leaflet coaptation-assist device includes a non-tissue-piercing anchor, and

wherein implanting the leaflet coaptation-assist device includes coupling the non-tissue-piercing anchor to a native cardiac structure.

Inventive Concept 175. The method according to Inventive Concept 174,

wherein the non-tissue-piercing anchor includes a stent, and

wherein coupling the non-tissue-piercing anchor to the native cardiac structure includes coupling the stent to atrial tissue by radial force and friction.

Inventive Concept 176. The method according to Inventive Concept 174,

wherein the non-tissue-piercing anchor includes one or more ventricular loops, and

wherein coupling the non-tissue-piercing anchor to the native cardiac structure includes coupling the one or more ventricular loops to a ventricular wall by radial force and friction.

Inventive Concept 177. The method according to Inventive Concept 176, wherein coupling the non-tissue-piercing anchor to the native cardiac structure includes bringing the one or more ventricular loops into contact with one or more of the leaflets at the commissural level and clipping the one or more leaflets within the one or more ventricular loops to stabilize anchoring of the leaflet coaptation-assist device.

There is still further provided, in accordance with an Inventive Concept 178 of the present invention, a method for promoting valve coaptation in a patient, the method including:

identifying incomplete coaptation of leaflets of a tricuspid valve of the patient, the leaflets selected from the group consisting of: native leaflets and prosthetic leaflets; and

implanting a leaflet coaptation-assist device that includes a sheet shaped so as to define three spokes extending radially outward from a central hub region, such that a coaptation-assist surface defined by a lower surface of the sheet is located at or below the leaflet hinge line and contacts an atrial side of the leaflets during systole, thereby enhancing leaflet coaptation.

Inventive Concept 179. The method according to Inventive Concept 178, wherein the three spokes are collectively shaped so as to define a deltoid curve.
Inventive Concept 180. The method according to Inventive Concept 178, wherein radially-outward ends of the spokes include respective stiffened portions.
Inventive Concept 181. The method according to Inventive Concept 178, wherein implanting the leaflet coaptation-assist device includes placing one or more ventricular loops in a ventricular chamber to stabilize the leaflet coaptation-assist device within the ventricular chamber.
Inventive Concept 182. The method according to Inventive Concept 178, wherein implanting the leaflet coaptation-assist device includes anchoring the leaflet coaptation-assist device in place using a plurality of tissue-piercing anchors.

The present invention will be more fully understood from the following detailed description of embodiments thereof, taken together with the drawings, in which:

### BRIEF DESCRIPTION OF THE DRAWINGS

FIGS. 1A-B are schematic illustrations of a leaflet coaptation-assist device, in accordance with an application of the present invention;

FIGS. 2A-C are schematic illustrations of another leaflet coaptation-assist device, in accordance with an application of the present invention;

FIG. 2D is a schematic illustration of the leaflet coaptation-assist device of FIGS. 2A-C implanted in a mitral valve, in accordance with an application of the present invention;

FIGS. 3A-D are schematic illustrations of still another leaflet coaptation-assist device, in accordance with an application of the present invention;

FIG. 3E is a schematic illustration of the leaflet coaptation-assist device of FIGS. 3A-D implanted in a mitral valve, in accordance with an application of the present invention;

FIG. 4 is a schematic illustration of yet another leaflet coaptation-assist device implanted in the mitral valve, in accordance with an application of the present invention;

FIGS. 5A-D are schematic illustrations of another leaflet coaptation-assist device, in accordance with an application of the present invention;

FIGS. 6A-B are schematic illustrations of still another leaflet coaptation-assist device, in accordance with an application of the present invention;

FIG. 6C is a schematic illustration of the leaflet coaptation-assist device of FIGS. 6A-B implanted in a mitral valve, in accordance with an application of the present invention;

FIG. 6D is a schematic illustration of a tissue anchor implanted in tissue of the native annulus, in accordance with an application of the present invention;

FIGS. 7A-C are schematic illustrations of still another leaflet coaptation-assist device, in accordance with an application of the present invention;

FIGS. 8 and 9 are schematic illustrations of additional leaflet coaptation-assist devices, in accordance with respective applications of the present invention;

FIGS. 10A-D are schematic illustrations of another leaflet coaptation-assist device, in accordance with an application of the present invention;

FIGS. 10E-G are schematic illustrations of the leaflet coaptation-assist device of FIGS. 10A-D implanted in a mitral valve, in accordance with an application of the present invention;

FIGS. 11A-E are schematic illustrations of still another leaflet coaptation-assist device, in accordance with an application of the present invention;

FIGS. 12A-D are schematic illustrations of yet another leaflet coaptation-assist device, in accordance with an application of the present invention;

FIGS. 13A-D are schematic illustrations of another leaflet coaptation-assist device, in accordance with an application of the present invention;

FIGS. 14A-F are schematic illustrations of still another leaflet coaptation-assist device, in accordance with an application of the present invention;

FIG. 14G is a schematic illustration of the leaflet coaptation-assist device of FIGS. 14A-F implanted in the mitral valve, in accordance with an application of the present invention;

FIGS. 15A-B are schematic illustrations of yet another leaflet coaptation-assist device, in accordance with an application of the present invention; and

FIG. 15C is a schematic illustration of the leaflet coaptation-assist device of FIGS. 15A-B implanted in a tricuspid valve, in accordance with an application of the present invention.

### DETAILED DESCRIPTION OF APPLICATIONS

Embodiments of the present invention provide leaflet coaptation-assist devices and methods for promoting valve coaptation of leaflets of an atrioventricular valve of a heart of a patient. In general, these techniques enhance coaptation by placing a patch or barrier at or near the flexural level (i.e., the leaflet hinge line, demarcated by an annulus 10) of the atrioventricular valve. Typically, these techniques limit the natural leaflet movement to level at or less than the flexural level. These techniques may be used to improve coaptation of native leaflets or prosthetic leaflets of a prosthetic valve and/or reduce or prevent valve prolapse, thereby minimizing leakage.

For some applications, the leaflet coaptation-assist devices described herein are delivered in a percutaneous transcatheter delivery procedure, and are thus capable of assuming both compressed delivery and expanded operational configurations. Typically, for these applications, the leaflet coaptation-assist devices are configured to be self-expanding upon release from the delivery system at the target location. Alternatively, the leaflet coaptation-assist devices are delivered transapically, or during an open surgical procedure.

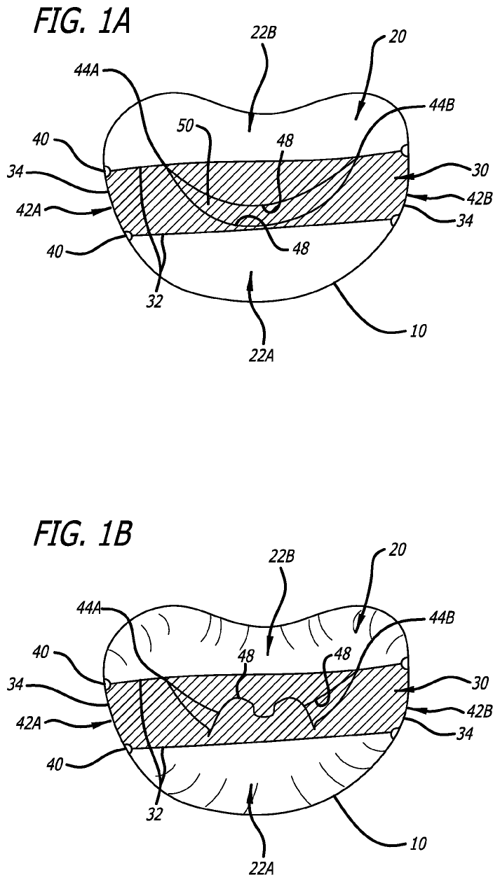

FIGS. 1A-B are schematic illustrations of a leaflet coaptation-assist device 20, in accordance with an application of the present invention. Leaflet coaptation-assist device 20 is configured to enhance leaflet coaptation of leaflets 22 (either native leaflets or prosthetic leaflets) of a mitral valve 24. Leaflets 22 include a posterior mitral leaflet 22A and an anterior mitral leaflet 22B. Mitral valve 24 is shown during diastole and systole in FIGS. 1A and 1B, respectively. In FIG. 1B, posterior mitral leaflet 22A is shown as prolapsing above anterior mitral leaflet 22B. Of course, as used herein, “systole” means “ventricular systole,” and “diastole” means “ventricular diastole,” in accordance with standard usage in the art and as evident from the drawings.

Leaflet coaptation-assist device 20 comprises:
a sheet 30 (i.e., a broad, thin, generally planar piece of material) having two longer sides 32 and two shorter sides 34, and a lower surface that defines a coaptation-assist surface; and
one or more tissue anchors 40, which are configured to anchor leaflet coaptation-assist device 20 in place within the heart, such that the two shorter sides 34 are disposed along two respective commissural areas 42A and 42B of annulus 10 of mitral valve 24 near respective commissures 44A and 44B of mitral valve 24, and the coaptation-assist surface is located at or below the leaflet hinge line and contacts respective atrial sides 46 of leaflets 22 during systole, such that both of leaflets 22 coapt with the coaptation-assist surface during systole.

For some applications, leaflet coaptation-assist device 20 is implanted such that, during systole, respective free edges 48 of leaflets 22 coapt with the coaptation-assist surface along entire lengths of free edges 48, such that the coaptation-assist surface entirely covers an orifice 50 of mitral valve 24.

Alternatively, for some applications, the leaflet coaptation-assist device is implanted such that, during systole, (a) a first free edge 48 of a first one of leaflets 22 coapts with the coaptation-assist surface along a majority of a first length of the first free edge 48, and (b) a second free edge 48 of a second one of leaflets 22 coapts with the coaptation-assist surface along a majority of a second length of the second free edge 48. Optionally, the leaflet coaptation-assist device is implanted such that, during systole, the first free edge 48 of the first one of leaflets 22 coapts with the coaptation-assist surface along an entirety of the first length of the first free edge 48 (and the second free edge 48 of the second one of leaflets 22 coapts with the coaptation-assist surface along a majority, but not an entirety, of the second length of the second free edge 48).

Leaflet coaptation-assist device 20 thus limits the movement of the native, and or other mitral assist device, to the level of placement of the device.

For example, for treating degenerative mitral valve regurgitation, leaflet coaptation-assist device 20 may block motion of leaflets 22 into the atrium and prevent leaflet prolapse; for such treatment, leaflet coaptation-assist device 20 is typically, although not necessarily, implanted at the leaflet hinge line. In addition, for example, for treating functional mitral valve regurgitation, leaflet coaptation-assist device 20 may provide a surface against which leaflets 22 partially coapt during systole so as to fill the area of non-coaptation between leaflets 22; for such treatment, leaflet coaptation-assist device 20 is typically, although not necessarily, implanted such that the coaptation surface thereof is below the leaflet hinge line, e.g., at least 2 mm, no more than 15 mm, and/or between 2 and 15 mm below the leaflet hinge line.

Optionally, sheet 30 has a thickness of less than 0.5 mm.

For some applications, sheet 30 is less flexible in two regions near the respective two shorter sides 34 than in a central region of the sheet.

In some applications of the present invention, a method is provided for promoting valve coaptation in a patient. The method comprises identifying incomplete coaptation of leaflets 22 of mitral valve 24, the leaflets 22 selected from the group consisting of: native leaflets and prosthetic leaflets. Leaflet coaptation-assist device 20 is implanted, the device including sheet 30 having the two longer sides 32 and the two shorter sides 34, such that the two shorter sides 34 are disposed along two respective commissural areas 42A and 42B of annulus 10 of mitral valve 24 near respective commissures 44A and 44B of mitral valve 24, and the coaptation-assist surface defined by the lower surface of sheet 30 is located at or below the leaflet hinge line and contacts respective atrial sides of leaflets 22 during systole, such that both of leaflets 22 coapt with the coaptation-assist surface during systole.

For some applications, sheet 30 has exactly four sides (as shown), e.g., has the shape of a quadrilateral (as shown), e.g., is rectangular (as shown) (rectangular sheet 30 does not appear entirely rectangle in FIGS. 1A-B because it has been deformed by attachment to the annulus). For some of these applications, tissue anchors 40 may be located at the four corners of sheet 30, such as shown. This enhances coaptation of the valve by forcing better coaptation and covering areas of poor coaptation.

Tissue anchors 40 may comprise screws, stents, stapes, sutures, loops, or any other suitable tissue anchors known in the art.

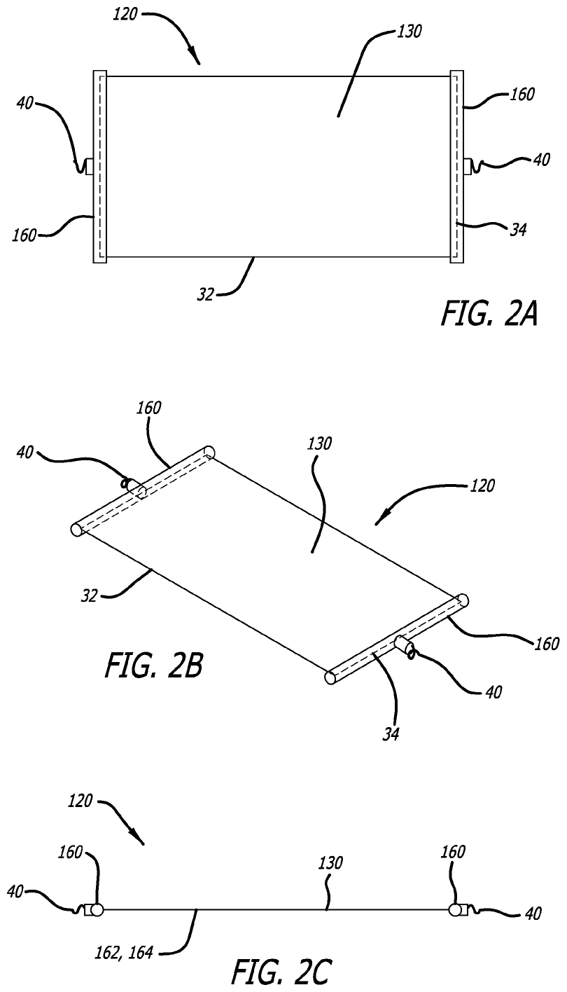

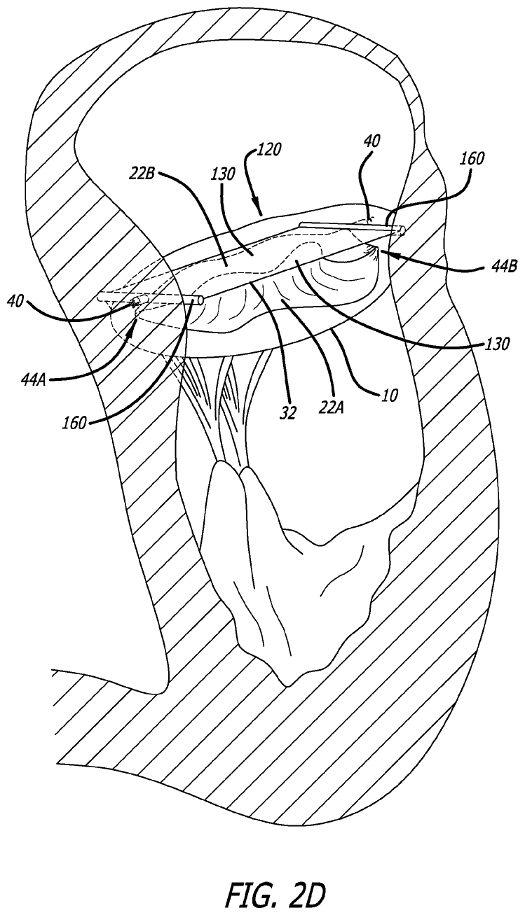

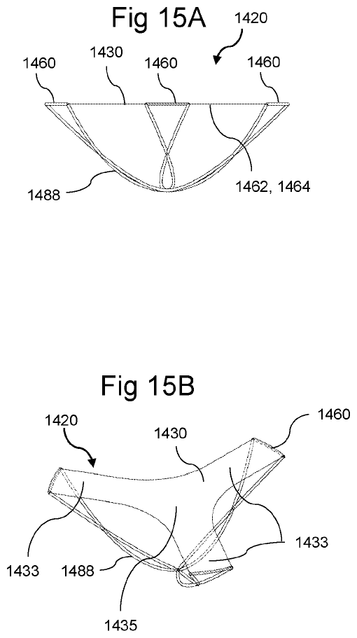

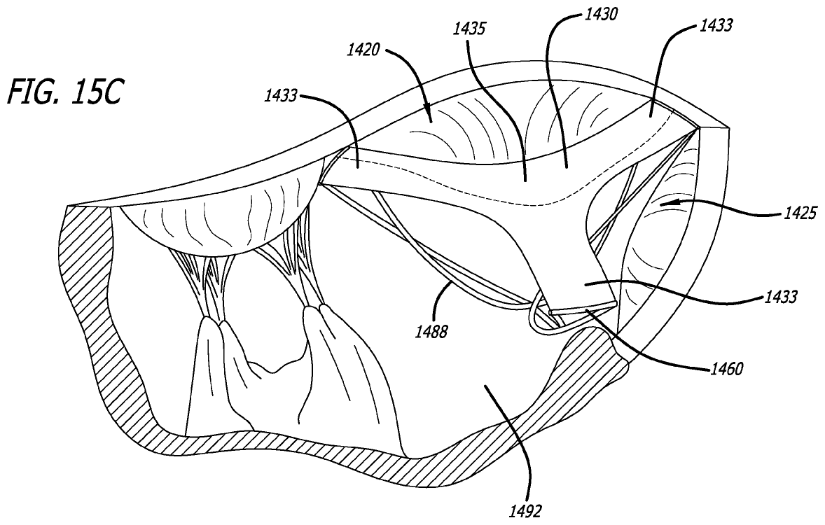

FIGS. 2-15C are schematic illustrations of leaflet coaptation-assist devices, in accordance with respective applications of the present in invention. Each of these leaflet coaptation-assist devices may be similar in some respects to leaflet coaptation-assist device 20, described hereinabove with reference to FIGS. 1A-B, and may implement any of the features thereof, mutatis mutandis. The features of the leaflet coaptation-assist devices illustrated in FIGS. 1A-B and 2-15C may be implemented in any combination. Although the leaflet coaptation-assist devices of FIGS. 1A-B and 2-14G are described and/or shown as treating the mitral valve, the leaflet coaptation-assist devices may also treat the tricuspid valve, the other atrioventricular valve, mutatis mutandis, as described hereinbelow, for example, with reference to FIGS. 15A-C.

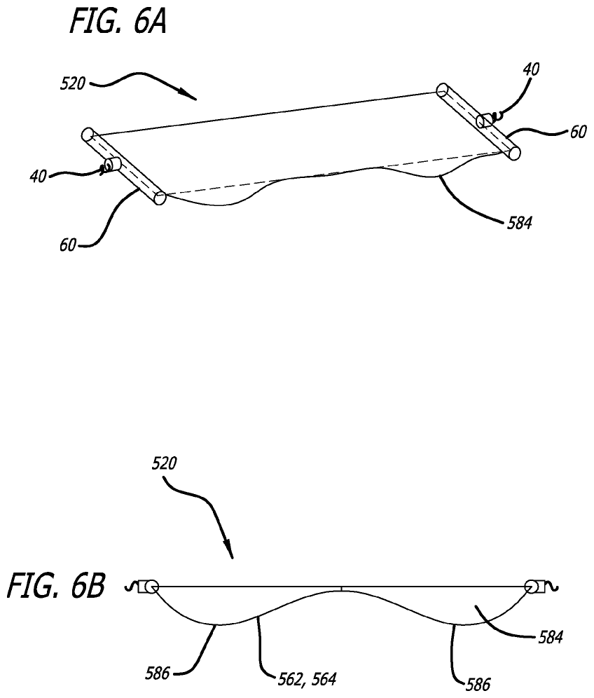

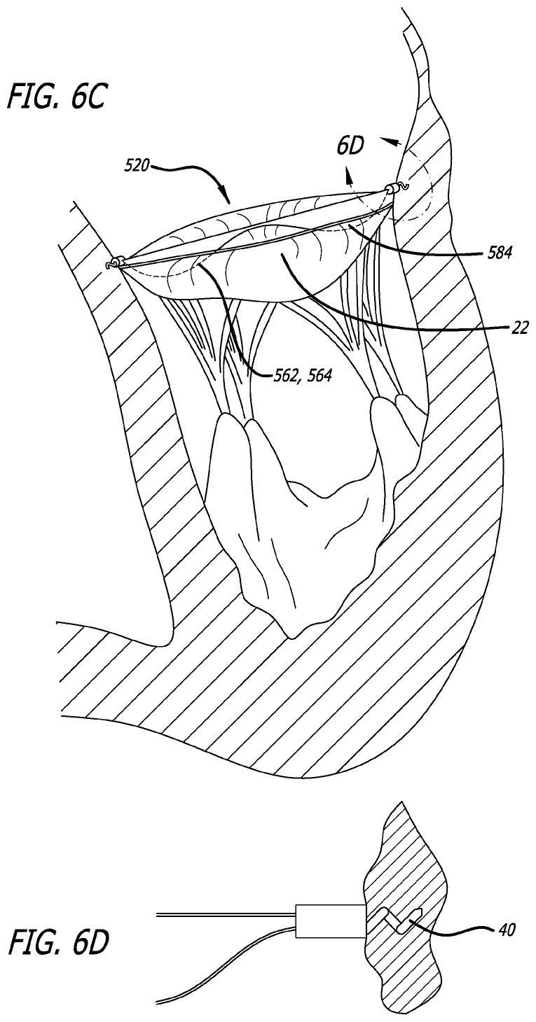

Each of the leaflet coaptation-assist devices comprises a coaptation-assist surface, which may, for example, be provided by a lower surface of a sheet, such as shown in FIGS. 2A-D, 3A-E, 4, 5A-D, 10A-G, 11A-E, 12A-D, 13A-D, 14A-G, and 15A-C, or a lower surface of a curved thick structure, such as a balloon or solid foam, such as shown in FIGS. 6A-D, 7A-C, 8, and 9. For some applications, the sheets comprise one or more of the following materials: biological tissue (e.g., pericardium), silicone, PEEK, silk, perylene, polyethylene, polyester, PVDF membrane, foam (e.g., polyurethane foam), a plastic material, rubber, latex, ePTFE, polyethylene, PET, nylon, or a metal (e.g., braided wires or tiny plate of nitinol) covered or coated. For some applications, the thick structure is covered with one or more of the above-listed materials.

Typically, the material of sheet 30 is flexible (rather than rigid), and optionally is floppy, i.e., generally does not retain its shape when not constrained to do so. However, the stiffened portions described below (e.g., stiffened portions 160), if formed from the sheet, are typically rigid or bendable.

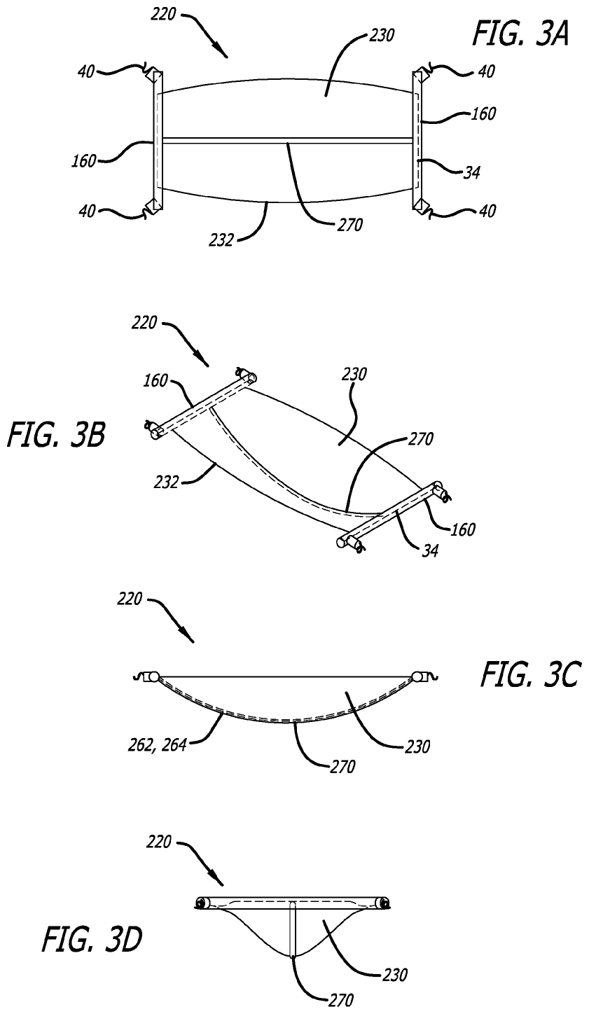

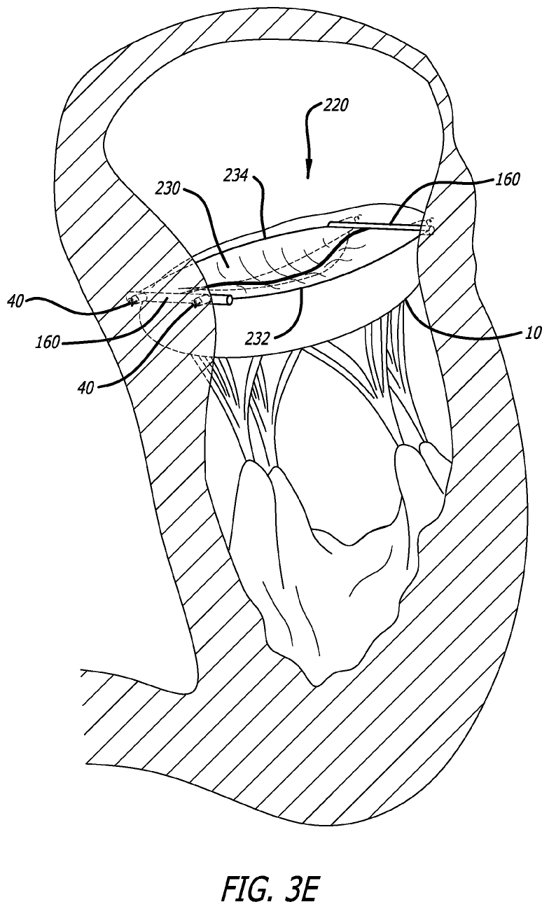

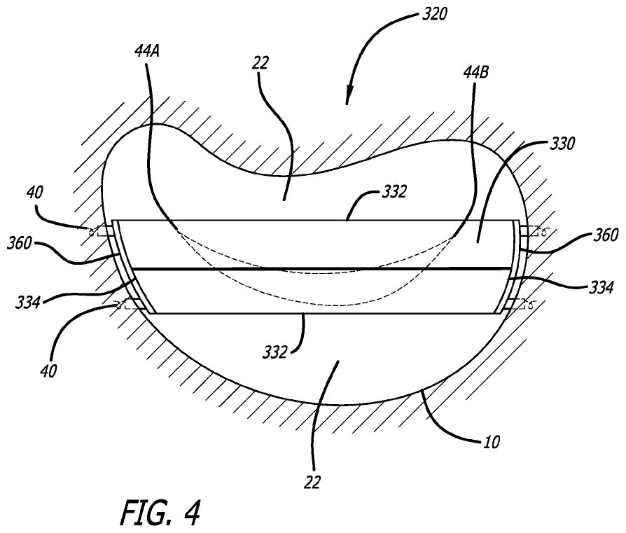

For some applications, sheets of the leaflet coaptation-assist devices of FIGS. 1A-B and 2-14G have four sides. For example, the sheets of the leaflet coaptation-assist devices may:
have the shape of a quadrilateral (four straight sides), e.g., a rectangle, such as shown in FIGS. 1A-B, 2A-D, 10A-G, 11A-E, 12A-D, 13A-D, and 14A-G;
have two straight sides and two curved sides (e.g., (a) two straight shorter sides and two curved longer sides, such as shown in FIGS. 3A-E and 5A-D, or (b) two straight longer sides and two curved shorter sides, such as shown in FIG. 4;
have four curved sides (configuration not shown); or
be shaped so as to define three spokes 1433 extending radially outward from a central hub region 1435, which may collectively have a deltoid curve shape (e.g., three straight short sides and three curved longer sides, such as shown in FIGS. 15A-C).

For other applications, the leaflet coaptation-assist devices have three sides or five or more sides (e.g., five to eight sides) (configurations not shown).

For some applications, the leaflet coaptation-assist devices of FIGS. 1A-B and 2-9 comprise a plurality of tissue-piercing anchors, which are optionally fixed to the sheet or thick structure, typically along shorter sides of the sheet or thick structure. The tissue-piercing anchors are configured to be coupled to the annulus of native valve or an annuloplasty ring (such as a rigid or semi-rigid ring, or a band) implanted around all or a portion of the native annulus, preferably at the leaflets commissural level. For example, the tissue-piercing anchors may comprise respective helical tissue-piercing coupling-elements, as shown.

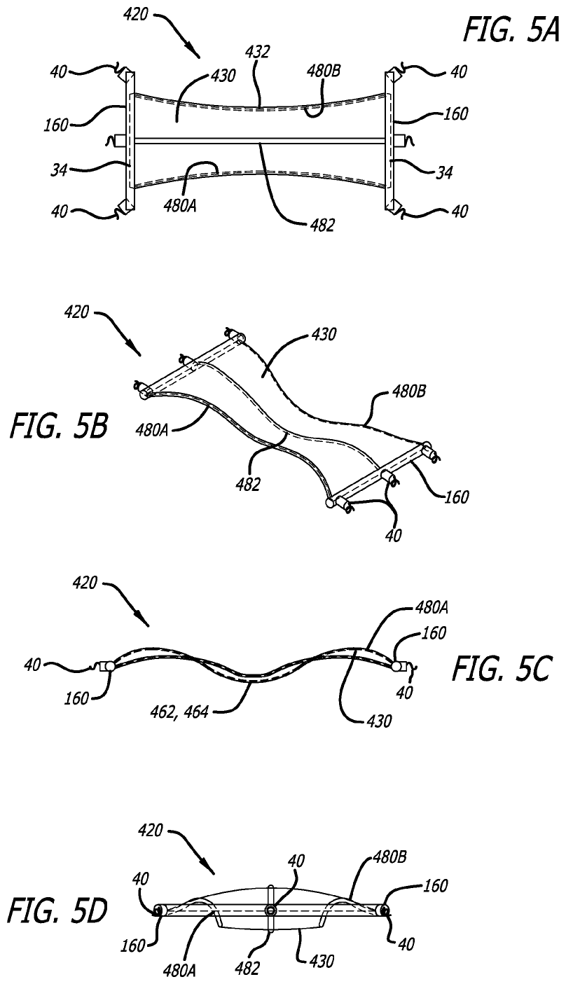

For example, the tissue-piercing anchors may be fixed to the sheet:
at or near the centers of the shorter sides of the sheet or leaflet coaptation-assist device, such as shown in FIGS. 2A-D, 6A-D, and 7A-C;
at or near the ends of the shorter sides of the sheet, such as shown in FIGS. 1A-B, 3A-E, and 4, in which case the leaflet coaptation-assist device comprises at least four tissue-piercing anchors; and
both at or near the centers of the shorter sides and at or near the ends of the shorter sides, such as shown in FIGS. 5A-D, in which case the leaflet coaptation-assist device comprises at least six tissue-piercing anchors.

Alternatively, in any of the configurations described hereinabove and hereinbelow, the tissue-piercing anchors are not fixed to the leaflet coaptation-assist device, but rather are separate and discrete from the leaflet coaptation-assist device, and are coupled to the sheet in situ during an implantation procedure.

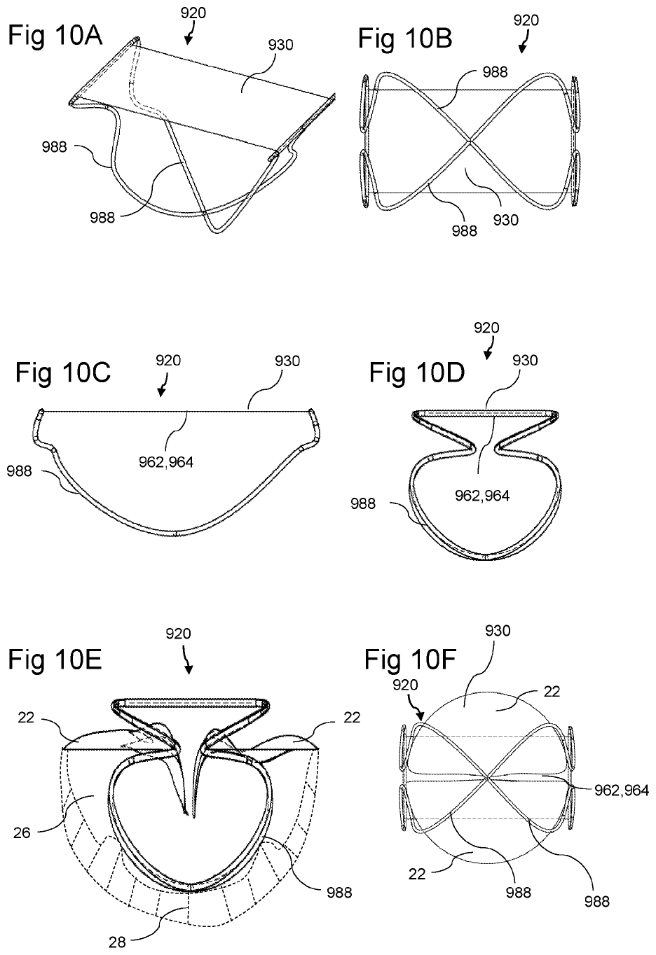

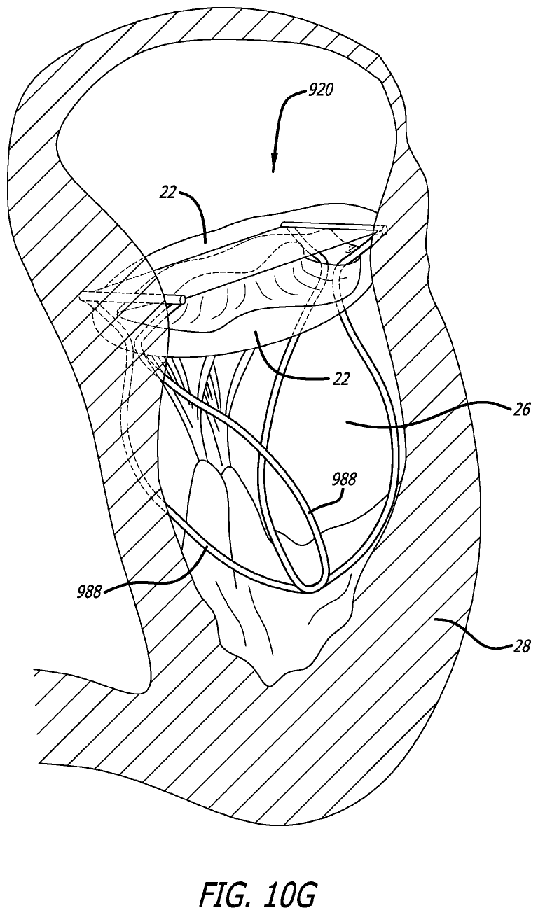

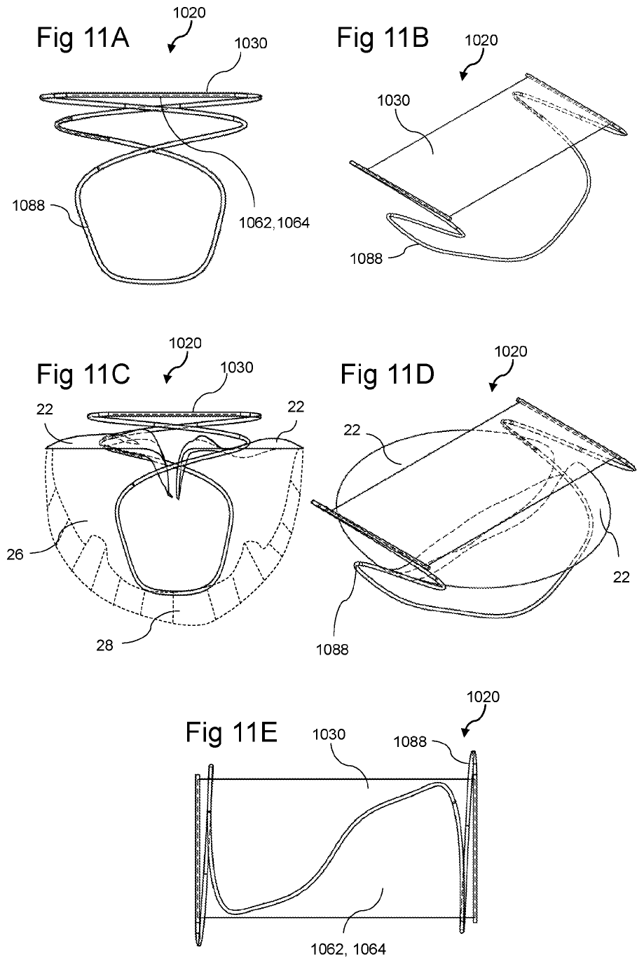

Further alternatively, in some configurations, the leaflet coaptation-assist devices comprise a non-tissue-piercing anchor, such as described hereinbelow with reference to FIGS. 10A-G, 11A-E, 12A-D, 13A-D, 14A-G, and 15A-C.

FIGS. 2A-C are schematic illustrations of a leaflet coaptation-assist device 120, in accordance with an application of the present invention, and FIG. 2D is a schematic illustration of leaflet coaptation-assist device 120 implanted in mitral valve 24, in accordance with an application of the present invention. In this configuration, leaflet coaptation-assist device 120 comprises a sheet 130, a lower surface 162 of which provides a coaptation-assist surface 164.

For some applications, as illustrated, the two shorter sides 34 of sheet 130 include respective stiffened portions 160 (which are stiffer than the remainder of sheet 130), which may be formed by two layers of sheet 130, or a folding of sheet 130, and/or comprise stiffener elements, which stabilize the leaflet coaptation-assist device against the annular tissue or annuloplasty ring. The stiffener elements may comprise of a polymer, a superelastic material, or a combination of such materials. Optionally, the stiffener elements comprise stitching.

Stiffened portions 160 help maintain the shape of sheet 130 (e.g., the rectangular shape, as shown), and allow the optional use of only a single tissue anchor 40 at each shorter side 34 rather than two or more anchors 40 to maintain the shape of sheet 130.

For some applications, the two longer sides 32 of sheet 130 have a length of at least 2 cm, no more than 6 cm, and/or between 2 and 6 cm, and/or the two shorter sides 34 of sheet 130 have a length of at least 0.5 cm, no more than 3 cm, and/or between 0.5 and 3 cm. Alternatively or additionally, for some applications, a ratio of the length of the longer sides 32 to the length of the shorter sides 34 is at least 2:1, no more than 5:1, and/or between 2:1 and 5:1.

For some applications, tissue anchors 40 are coupled to sheet 130 via the respective stiffened portions 160, such as shown.

FIGS. 3A-D are schematic illustrations of a leaflet coaptation-assist device 220, in accordance with an application of the present invention, and FIG. 3E is a schematic illustration of leaflet coaptation-assist device 120 implanted in mitral valve 24, in accordance with an application of the present invention. In this configuration, leaflet coaptation-assist device 220 comprises a sheet 230 having a ventricular (lower) surface 262 that provides a coaptation-assist surface 264. Sheet 230 is elliptically curved with a curvature shape toward the ventricular (lower) surface; in other words, lower surface 262 of sheet 230 has an elliptically convex curvature. The curvature provides multiple planes of coaptation for leaflets 22, rather than a single plane along the implantation plane.

For example, the curved shape (elliptically convex curvature) may be provided by a curved stiffener 270 coupled to (e.g., embedded within) sheet 230. For example, curved stiffener 270 may comprise a material having a shape memory, such as a metal (e.g., Nitinol) or a polymer.

For some applications, leaflet coaptation-assist device 220 comprises two tissue-piercing anchors 40 at or near the ends of each shorter side 34 of sheet 230; alternatively, leaflet coaptation-assist device 220 comprises fewer or more tissue-piercing anchors 40, or no tissue-piercing anchors (for example, as described hereinbelow with reference to FIGS. 10A-G, 11A-E, 12A-D, 13A-D, 14A-G, and 15A-C, mutatis mutandis). Sheet 230 may have two straight shorter sides 34 and two curved longer sides 232 (such as shown); the excess material of the curvatures may help fully cover orifice 50 of mitral valve 24. Alternatively, sheet 230 may be rectangular (configuration not shown, but similar to the configuration shown in FIGS. 2A-D).

FIG. 4 is a schematic illustration of a leaflet coaptation-assist device 320 implanted in mitral valve 24, in accordance with an application of the present invention. In this configuration, leaflet coaptation-assist device 320 comprises a sheet 330 having curved shorter sides 334 and straight longer sides 332, and curved stiffened portions 360 corresponding the curvature of curved shorter sides 334. Stiffened portions 360 may implement any of the features of stiffened portions 160, described hereinabove with reference to FIGS. 2A-D, mutatis mutandis. Alternatively, longer sides 332 are also curved, like longer sides 232 described hereinabove with reference to FIGS. 3A-E.

Alternatively, in another configuration, shorter sides 334 are straight when in a relaxed, resting configuration, and are bendable by forces applied during implantation, in order to assume the curved configurations shown in FIG. 4.

Optionally, sheet 330 is elliptically curved, similar to elliptic curved sheet 230, described hereinabove with reference to FIGS. 3A-E.

FIGS. 5A-D are schematic illustrations of a leaflet coaptation-assist device 420, in accordance with an application of the present invention. In this configuration, leaflet coaptation-assist device 420 comprises a concave multi-curved sheet 430, with two rounded convex curvature shapes lateral to sheet 430, near shorter sides 34 of sheet 430. A ventricular (lower) surface 462 of sheet 430 provides a coaptation-assist surface 464. In other words, lower surface 462 of sheet 430 has a bi-lobal convex curvature having two rounded curvature shapes. The curvature provides multiple planes of coaptation for leaflets 22, rather than a single plane along the implantation plane.

The bi-lobal shape may be provided by one or more (e.g., one, two, or three) curved stiffeners 480A, 480B, and/or 482 coupled to (e.g., embedded within) sheet 430. For example, the curved stiffeners may comprise a material having a shape memory, such as a metal (e.g., Nitinol) or a polymer. For example, the one or more curved stiffeners may include two edge curved stiffeners 480A and 480B that run along longer sides 432 of sheet 430, and/or a center curved stiffener 482 that runs along a longer centerline of sheet 430, as shown.

For some applications, leaflet coaptation-assist device 420 comprises three tissue-piercing anchors 40, of which two are at or near the ends of each shorter side 34 of sheet 430 and one is at or near the center of each shorter side 34 of sheet 430; alternatively, leaflet coaptation-assist device 420 comprises fewer or more tissue-piercing anchors 40, or no tissue-piercing anchors (for example, as described hereinbelow with reference to FIGS. 10A-G, 11A-E, 12A-D, 13A-D, 14A-G, and 15A-C, mutatis mutandis).

FIGS. 6A-B are schematic illustrations of a leaflet coaptation-assist device 520, in accordance with an application of the present invention, and FIG. 6C is a schematic illustration of leaflet coaptation-assist device 520 implanted in mitral valve 24, in accordance with an application of the present invention. In this configuration, leaflet coaptation-assist device 520 comprises a thick structure 584 having a lower surface 562 that provides a coaptation-assist surface 564. In order to provide its thickness, thick structure 584 may comprise, for example, a solid foam or at least one balloon, which may be inflated with a liquid, such as water, saline, contrast media, glue, foam, and/or blood. As used in the present application, including the claims, the word “solid” in “solid foam” refers to the state of matter, rather than implying any level of rigidity of the foam. For some applications, the solid foam comprises a polymer, such as polyurethane or silicone.

For some applications, thick structure 584 is curved, such as multi-curved, on ventricular (lower) surface 562, such as with a bi-lobal shape 586 provided by the body of thick structure 584. The curvature provides multiple planes of coaptation for leaflets 22, rather than a single plane along the implantation plane.

Optionally, thick structure 584 is planar on the upper (atrial) side.

Optionally, thick structure 584 of leaflet coaptation-assist device 520 has a greatest thickness (typically near the two shorter edges (i.e., near the commissures)) in the atrial-ventricular direction of at least 2 mm (e.g., at least 3 mm), no more than 20 mm, and/or between 2 and 20 mm, e.g., between 3 and 20 mm, and/or a smallest thickness (typically near the center) in the atrial-ventricular direction of at least 1 mm, no more than 5 mm, and/or between 1 and 5 mm.

FIG. 6D is a schematic illustration of one of tissue anchors 40 implanted in tissue of the native annulus, in accordance with an application of the present invention.

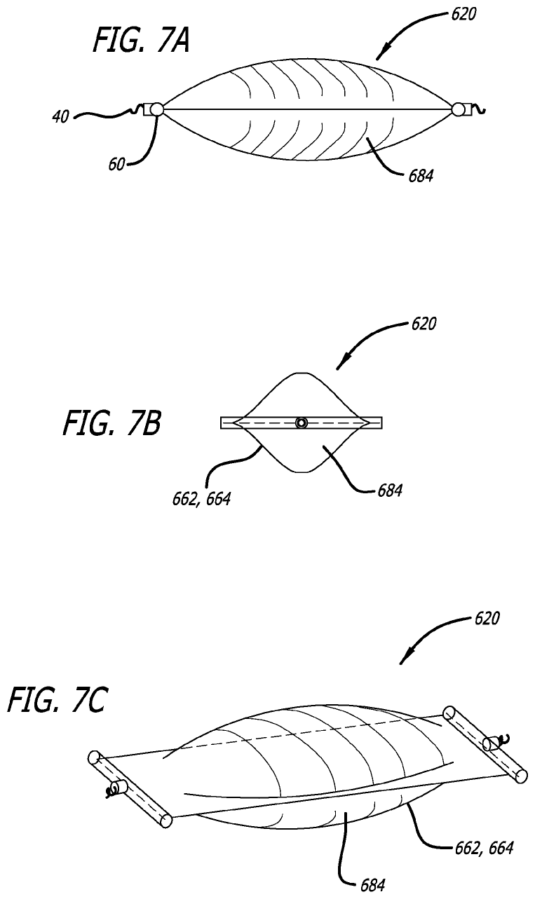

FIGS. 7A-C are schematic illustrations of a leaflet coaptation-assist device 620, in accordance with an application of the present invention. In this configuration, leaflet coaptation-assist device 420 comprises a convex thick structure 684, with an atrial and ventricular curved shape provided by the body of thick structure 684. In order to provide its thickness, thick structure 684 may comprise, for example, a solid foam or at least one balloon, which may be inflated with a liquid, such as water, saline, contrast media, glue, foam, and/or blood. A ventricular (lower) surface 662 of thick structure 684 provides a coaptation-assist surface 664. The curvature provides multiple planes of coaptation for leaflets 22, rather than a single plane along the implantation plane. Leaflet coaptation-assist device 620 may have any of the features of leaflet coaptation-assist device 520, described hereinabove with reference to FIGS. 6A-D.

Optionally, thick structure 684 of leaflet coaptation-assist device 620 has a greatest thickness (typically near the center) in the atrial-ventricular direction of at least 6 mm, no more than 40 mm, and/or between 6 and 40 mm, and/or a smallest thickness (typically near the two shorter edges (i.e., near the commissures)) in the atrial-ventricular direction of at least 1 mm, no more than 5 mm, and/or between 1 and 5 mm.

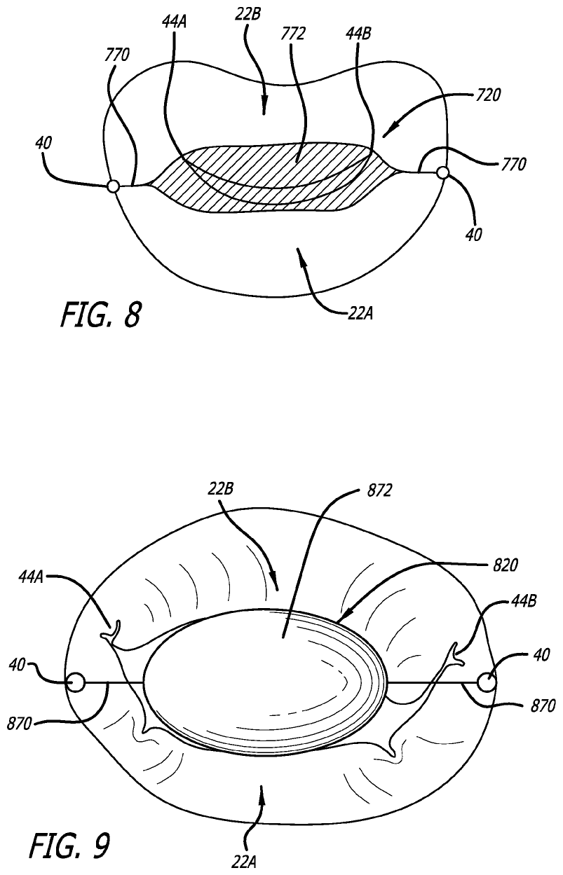

FIGS. 8 and 9 are schematic illustrations of leaflet coaptation-assist devices 720 and 820, respectively, in accordance with respective applications of the present invention. Leaflet coaptation-assist device 720 and 820 comprise volume filling components. As shown in FIG. 8, the volume filling component of leaflet coaptation-assist device 720 comprises a spindle bead 772, and as shown in FIG. 9, the volume filling component of leaflet coaptation-assist device 720 comprises a spherical shaped bead 872. Such volume filling components are anchored on tethers 770 and 780, respectively, which may be coupled to tissue of the heart by anchoring techniques described herein.

For some applications, the volume filling components comprise elastomeric balloons, which are configured to be inflated in situ. For other applications, the volume filling components are self-swelling upon delivery.

Leaflet coaptation-assist device 720 may further comprise sheet 30 and/or tissue anchors 40, such as described hereinabove with reference to FIGS. 1A-B, mutatis mutandis. Leaflet coaptation-assist device 820 may further comprise tissue anchors 40, such as described hereinabove with reference to FIGS. 1A-B, mutatis mutandis.

FIGS. 10A-D are schematic illustrations of a leaflet coaptation-assist device 920, in accordance with an application of the present invention, and FIGS. 10E-G are schematic illustrations of leaflet coaptation-assist device 920 implanted in mitral valve 24, in accordance with an application of the present invention. In this configuration, leaflet coaptation-assist device 920 may comprise a sheet 930 (as shown), which may implement any of the sheet configurations described herein, or a thick structure (configuration not shown), which may implement any of the thick structure configurations described herein. In either case, a ventricular (lower) surface 962 of the sheet or thick structure provides a coaptation-assist surface 964.

In this configuration, leaflet coaptation-assist device 920 comprises a non-tissue-piercing anchor, which may comprise one or more (e.g., two or more) ventricular loops 988, which optionally comprises a superelastic material. The one or more ventricular loops 988 extend in a ventricular direction from sheet 930 or thick structure, crossing the native or prosthetic valve in the commissural area. For some applications, the one or more ventricular loops 988 are fixed to the shorter sides of sheet 930 or thick structure. For some applications, the one or more ventricular loops 988 stabilize the device by friction and radial force.

Optionally, in configurations in which leaflet coaptation-assist device 920 comprises two or more ventricular loops 988, the two or more ventricular loops 988 cross one other, with the crossing located within a ventricular chamber 26 upon implantation of leaflet coaptation-assist device 920, such as shown in FIGS. 10A-G.

FIGS. 10E-G show leaflet coaptation-assist device 920 implanted in mitral valve 24, with leaflets 22 below the coaptation-assist device, and with the one or more ventricular loops 988 extending within ventricular chamber 26, extending to a ventricular apical area 28.

FIGS. 11A-E are schematic illustrations of a leaflet coaptation-assist device 1020, in accordance with an application of the present invention. Other than as described below, leaflet coaptation-assist device 1020 is similar to leaflet coaptation-assist device 920, described hereinabove with reference to FIGS. 10A-G. A ventricular (lower) surface 1062 of a sheet 1030 or thick structure provides a coaptation-assist surface 1064. In this configuration, leaflet coaptation-assist device 1020 comprises a non-tissue-piercing anchor that comprises a single ventricular loop 1088, which is optionally fixed to both the shorter sides of sheet 1030 or the thick structure. For some applications, such as shown in FIG. 11D, ventricular loop 1088 is configured to come into contact with one of leaflets 22 at the commissural level and clip the leaflet within ventricular loop 1088 to stabilize the anchoring of leaflet coaptation-assist device 1020.

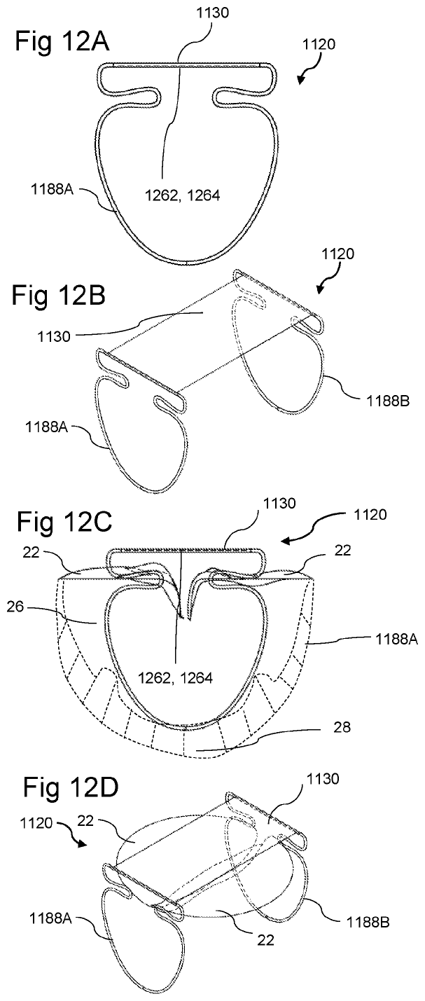

FIGS. 12A-D are schematic illustrations of a leaflet coaptation-assist device 1120, in accordance with an application of the present invention. Other than as described below, leaflet coaptation-assist device 1120 is similar to leaflet coaptation-assist device 920, described hereinabove with reference to FIGS. 10A-G. A ventricular (lower) surface 1162 of a sheet 1130 or thick structure provides a coaptation-assist surface 1164. In this configuration, leaflet coaptation-assist device 1120 comprises a non-tissue-piercing anchor that comprises two separate independent, non-crossing ventricular loops 1188A and 1188B, which do not touch one another, and which are typically configured to be positioned ventricularly below the commissural valve level. Typically, ventricular loops 1188A and 1188B are coupled to the shorter sides of sheet 1130 or the thick structure.

For some applications, such as shown in FIGS. 12C-D, ventricular loops 1188A and 1188B are configured to come into contact with both of leaflets 22 at the commissural level and clip leaflets 22 within the ventricular loops to stabilize the anchoring of leaflet coaptation-assist device 1120.

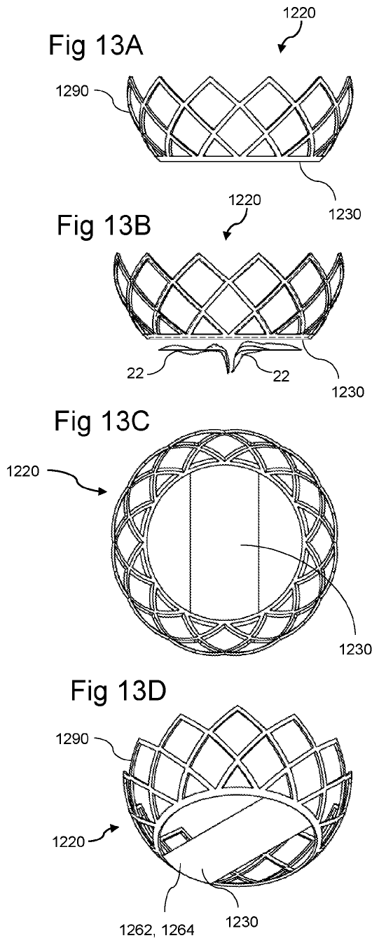

FIGS. 13A-D are schematic illustrations of a leaflet coaptation-assist device 1220, in accordance with an application of the present invention. In this configuration, leaflet coaptation-assist device 1220 may comprise a sheet 1230 (as shown), which may implement any of the sheet configurations described herein, or a thick structure (configuration not shown), which may implement any of the thick structure configurations described herein. In either case, a ventricular (lower) surface 1262 of sheet 1230 or the thick structure provides a coaptation-assist surface 1264. In this configuration, leaflet coaptation-assist device 1220 comprises a non-tissue-piercing anchor, which comprises a stent 1290, which is configured to be coupled to an atrial wall, so as to position leaflet coaptation-assist device 1220 at the annular level, at or below the leaflet hinge line of leaflets 22, optionally depending of the height of coupling of stent 1290 above the atrioventricular valve.

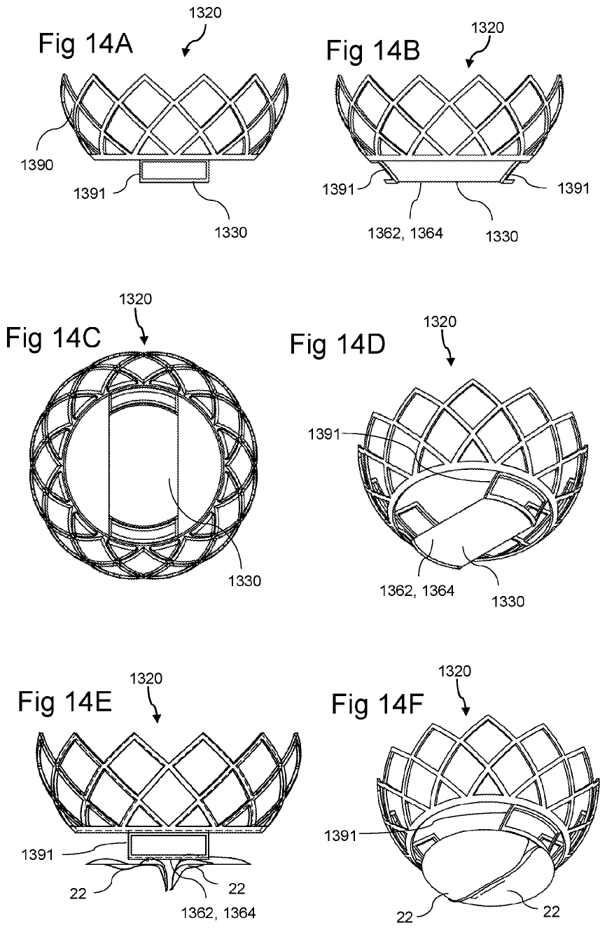

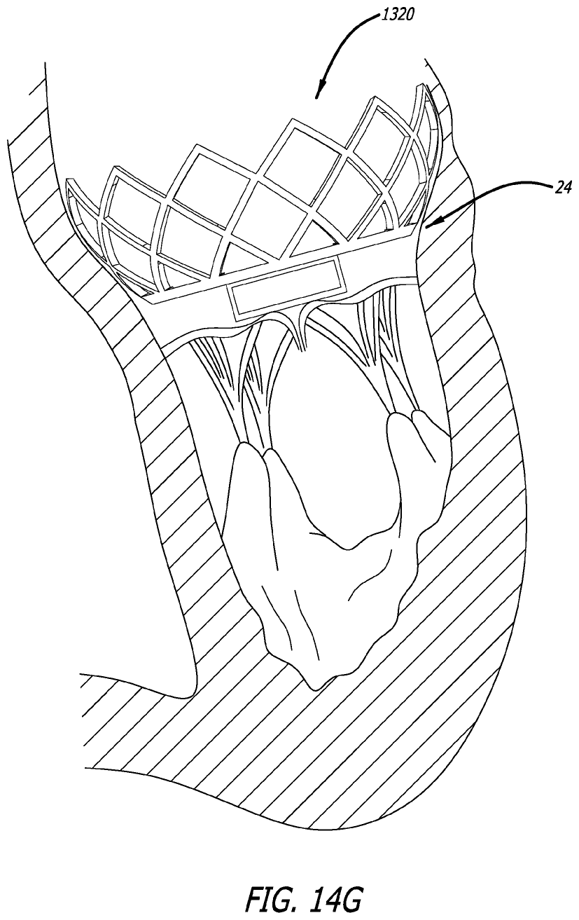

FIGS. 14A-F are schematic illustrations of a leaflet coaptation-assist device 1320, in accordance with an application of the present invention, and FIG. 14G is a schematic illustration of leaflet coaptation-assist device 1320 implanted in mitral valve 24, in accordance with an application of the present invention. Other than as described below, leaflet coaptation-assist device 1320 is similar to leaflet coaptation-assist device 1220, described hereinabove with reference to FIGS. 13A-D. A ventricular (lower) surface 1362 of a sheet 1330 or a thick structure provides a coaptation-assist surface 1364. In this configuration, leaflet coaptation-assist device 1320 is configured to position the sheet or thick structure below the annular level, so to favorite the below leaflet coaptation with coaptation-assist surface 1364. For example, a stent 1390 may comprise ventricular extensions 1391, which extend ventricularly to support sheet 1330 or the thick structure below the annular level.

FIGS. 15A-B are schematic illustrations of a leaflet coaptation-assist device 1420, in accordance with an application of the present invention, and FIG. 15C is a schematic illustration of leaflet coaptation-assist device 1420 implanted in a tricuspid valve 1425, in accordance with an application of the present invention. Leaflet coaptation-assist device 1420 is configured to treat tricuspid valve 1425 by promoting valve coaptation of the leaflets of tricuspid valve 1425. To this end, leaflet coaptation-assist device 1420 comprises a sheet 1430 that is shaped so as to define three spokes 1433 extending radially outward from a central hub region 1435. (It is to be understood that the word “spokes” does not imply that sheet 1430 further comprises a wheel at the outer ends of the spokes.) Alternatively, leaflet coaptation-assist device 1420 comprises a thick structure (configuration not shown), which may implement any of the thick structure configurations described herein in combination with the three-spoke shape. A ventricular (lower) surface 1462 of sheet 1430 or the thick structure provides a coaptation-assist surface 1464.

Typically, the three spokes 1433 are evenly distributed around the central hub region 1435, i.e., at an angle of 120 degrees with respect to one another. Optionally, sheet 1430 is rotationally symmetric, such as shown.

For some applications, the three spokes 1433 are collectively shaped so as to define a deltoid curve (e.g., three straight short sides and three curved longer sides).

For some applications, radially-outward ends of spokes 1433 include respective stiffened portions 1460, which may be formed as described hereinabove with reference to FIGS. 2A-D for stiffened portions 160.

Typically, the stiffened radial ends of sheet 1430 connect the sheet to the annular level.

For some applications, leaflet coaptation-assist device 1420 comprises a non-tissue-piercing anchor, which may comprise one or more (e.g., two or more) ventricular loops 1488, which optionally comprises a superelastic material. The one or more ventricular loops 1488 extend in a ventricular direction from sheet 1430 or the thick structure through the commissural spaces, and stabilize within a ventricular chamber 1492 and position the device at the leaflets coaptation level. Alternatively, for other applications, leaflet coaptation-assist device 1420 comprises tissue-piercing anchors, such as described hereinabove, or does not comprise any anchors.

In some applications of the present invention, a method is provided for promoting valve coaptation in a patient. The method comprises identifying incomplete coaptation of leaflets of tricuspid valve 1425, the leaflets selected from the group consisting of: native leaflets and prosthetic leaflets. Leaflet coaptation-assist device 1420 is implanted, the device including sheet 1430 shaped so as to define three spokes 1433 extending radially outward from central hub region 1435, such that coaptation-assist surface 1646 defined by lower surface 1642 of sheet 1430 is located at or below the leaflet hinge line and contacts an atrial side of the leaflets during systole, thereby enhancing leaflet coaptation.

Although the leaflet assist devices described herein are generally described in the context of an atrioventricular valve, they can be used to enhance the performance of any of the valves in the circulatory system, including a venous valve, e.g., a peripheral venous valve.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

In an embodiment, techniques and apparatus described in one or more of the following applications, which are assigned to the assignee of the present application and are incorporated herein by reference, are combined with techniques and apparatus described herein:
PCT Publication WO 2014/207575 to Schewel
PCT Publication WO 2018/142217 to Kuck et al.
PCT Publication WO 2020/148755 to Guidotti et al.
U.S. Provisional Application 61/956,683, filed Jun. 14, 2013
U.S. Provisional Application 61/963,330, filed Dec. 2, 2013
U.S. Provisional Application 61/982,307, filed Apr. 21, 2014
U.S. Provisional Application 62/455,427, filed Feb. 6, 2017
U.S. Provisional Application 62/570,336, filed Oct. 10, 2017
U.S. Provisional Application 62/792,092, filed Jan. 14, 2019
U.S. Provisional Application 62/884,404, filed Aug. 8, 2019

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1. A leaflet coaptation-assist device for promoting valve coaptation of leaflets of an atrioventricular valve of a heart of a patient, the leaflet coaptation-assist device comprising:
a coaptation-assist surface; and
one or more tissue anchors, which are configured to anchor the leaflet coaptation-assist device in place within the heart of the patient, such that the coaptation-assist surface is located at or below the leaflet hinge line and contacts an atrial side of the leaflets during ventricular systole, thereby enhancing leaflet coaptation, the leaflets selected from the group consisting of: native leaflets and prosthetic leaflets,
wherein the one or more tissue anchors comprise a non-tissue-piercing anchor, which comprises one or more ventricular loops, which are configured to couple the leaflet coaptation-assist device to a ventricular wall by radial force and friction.

2. The leaflet coaptation-assist device according to claim 1, wherein the one or more ventricular loops are configured to contact one or more of the atrioventricular leaflets at the commissural level and clip the one or more leaflets within the one or more ventricular loops to stabilize anchoring of the leaflet coaptation-assist device.

3. The leaflet coaptation-assist device according to claim 1, wherein the non-tissue-piercing anchor comprises a single ventricular loop.

4. The leaflet coaptation-assist device according to claim 1, wherein the non-tissue-piercing anchor comprises two or more ventricular loops.

5. The leaflet coaptation-assist device according to claim 4, wherein the two or more ventricular loops or cross one another.

6. The leaflet coaptation-assist device according to claim 4, wherein the two or more ventricular loops do not cross or touch one another.

7. The leaflet coaptation-assist device according to claim 1, wherein the one or more ventricular loops are configured to reach to a ventricular apical area.

8. The leaflet coaptation-assist device according to claim 1, wherein the atrioventricular valve is a mitral valve.

9. The leaflet coaptation-assist device according to claim 1, wherein the atrioventricular valve is a tricuspid valve.

10. The leaflet coaptation-assist device according to claim 1, wherein the one or more tissue anchors are configured to anchor the leaflet coaptation-assist device in place within the heart of the patient, such that the coaptation-assist surface is located at the leaflet hinge line and contacts the atrial side of the leaflets during ventricular systole, thereby blocking motion of the leaflets into an atrium and preventing leaflet prolapse.

11. The leaflet coaptation-assist device according to claim 1, wherein the one or more tissue anchors are configured to anchor the leaflet coaptation-assist device in place within the heart of the patient, such that the coaptation-assist surface is located below the leaflet hinge line such that the leaflets partially coapt with the coaptation-assist surface during ventricular systole.

12. The leaflet coaptation-assist device according to any one of claims 1-11, wherein the leaflet coaptation-assist device comprises a sheet having a lower surface that provides the coaptation-assist surface.

13. The leaflet coaptation-assist device according to claim 12, wherein the sheet has four sides.

14. The leaflet coaptation-assist device according to claim 13, wherein the sheet has a shape of a quadrilateral.

15. The leaflet coaptation-assist device according to claim 12, wherein the sheet is shaped so as to define three spokes extending radially outward from a central hub region.

16. The leaflet coaptation-assist device according to claim 15, wherein the three spokes are collectively shaped so as to define a deltoid curve.

17. The leaflet coaptation-assist device according to claim 13, wherein the sheet has two straight sides and two curved sides.

18. The leaflet coaptation-assist device according to claim 17, wherein the sheet has two straight shorter sides and two curved longer sides.

19. The leaflet coaptation-assist device according to claim 17, wherein the sheet has two straight longer sides and two curved shorter sides.

20. The leaflet coaptation-assist device according to claim 12, wherein the sheet has two longer sides and two shorter sides, and the shorter sides include respective stiffened portions.

21. The leaflet coaptation-assist device according to any one of claims 1-11, wherein the leaflet coaptation-assist device comprises a thick structure having a lower surface that provides the coaptation-assist surface.

22. The leaflet coaptation-assist device according to claim 21, wherein the thick structure comprises a solid foam.

23. The leaflet coaptation-assist device according to claim 21, wherein the thick structure comprises at least one balloon.

24. The leaflet coaptation-assist device according to claim 21, wherein the thick structure is curved on a ventricular side thereof.

25. The leaflet coaptation-assist device according to claim 24, wherein the thick structure is multi-curved on the ventricular side thereof.

26. The leaflet coaptation-assist device according to claim 21, wherein the thick structure is flat on an atrial side thereof.

27. The leaflet coaptation-assist device according to claim 21, wherein the thick structure has a greatest thickness in the atrial-ventricular direction of at least 3 mm.

28. The leaflet coaptation-assist device according to claim 27, wherein the thick structure has a greatest thickness in the atrial-ventricular direction of no more than 40 mm.

29. A leaflet coaptation-assist device for promoting valve coaptation of leaflets of a mitral valve of a heart of a patient, the leaflet coaptation-assist device comprising:
a sheet having a lower surface that defines a coaptation-assist surface, and two longer sides and two shorter sides, the shorter sides including respective stiffened portions; and
a plurality of tissue-piercing tissue anchors, which are configured to be coupled to a structure selected from the group consisting of: an annulus of the mitral valve and an annuloplasty ring implanted on the annulus of the mitral valve, so as to anchor the leaflet coaptation-assist device in place within the heart of the patient, such that the coaptation-assist surface is located at or below the leaflet hinge line and contacts an atrial side of the leaflets during systole, thereby enhancing leaflet coaptation, the leaflets selected from the group consisting of: native leaflets and prosthetic leaflets.

30. The leaflet coaptation-assist device according to claim 29, wherein the tissue anchors are configured to anchor the leaflet coaptation-assist device in place within the heart of the patient, such that the coaptation-assist surface is located at the leaflet hinge line and contacts the atrial side of the leaflets during systole, thereby blocking motion of the leaflets into an atrium and preventing leaflet prolapse.

31. The leaflet coaptation-assist device according to claim 29, wherein the tissue anchors are configured to anchor the leaflet coaptation-assist device in place within the heart of the patient, such that the coaptation-assist surface is located below the leaflet hinge line such that the leaflets partially coapt with the coaptation-assist surface during systole.

32. The leaflet coaptation-assist device according to claim 29, wherein each of the stiffened portions is formed by two layers of the sheet or by a folding of the sheet.

33. The leaflet coaptation-assist device according to claim 29, wherein the stiffened portions comprise respective stiffener elements.

34. The leaflet coaptation-assist device according to claim 29, wherein the tissue anchors are configured to anchor the leaflet coaptation-assist device to an annulus of the mitral valve.

35. The leaflet coaptation-assist device according to claim 29, wherein the tissue anchors are configured to anchor the leaflet coaptation-assist device to an annuloplasty ring implanted on an annulus of the mitral valve.

36. The leaflet coaptation-assist device according to claim 29, wherein the sheet has exactly four sides.

37. The leaflet coaptation-assist device according to claim 36, wherein the sheet has a shape of a quadrilateral.

38. The leaflet coaptation-assist device according to claim 36, wherein the sheet has two straight sides and two curved sides.

39. The leaflet coaptation-assist device according to claim 38, wherein the two shorter sides are straight and the two longer sides are curved.

40. The leaflet coaptation-assist device according to claim 38, wherein the two longer sides are straight and the shorter sides are curved.

41. The leaflet coaptation-assist device according to claim 29, wherein the tissue-piercing anchors comprise respective helical tissue-piercing coupling-elements.

42. The leaflet coaptation-assist device according to any one of claims 29-41, wherein the lower surface of the sheet has an elliptically convex curvature.

43. The leaflet coaptation-assist device according to claim 42, further comprising a curved stiffener that is coupled to the sheet and provides the elliptically convex curvature.

44. The leaflet coaptation-assist device according to any one of claims 29-41, wherein the lower surface of the sheet has a bi-lobal convex curvature having two rounded curvature shapes.

45. The leaflet coaptation-assist device according to claim 44, further comprising one or more curved stiffeners that are coupled to the sheet and provide the bi-lobal convex curvature.

46. The leaflet coaptation-assist device according to claim 43, wherein the curved stiffener comprises a material having a shape memory.

47. The leaflet coaptation-assist device according to any one of claims 29-41, wherein the tissue-piercing anchors are fixed to the coaptation-assist surface along the two shorter sides of the sheet.

48. The leaflet coaptation-assist device according to claim 47, wherein the tissue-piercing anchors are fixed to the coaptation-assist surface at or near respective centers of the two shorter sides of the sheet.

49. The leaflet coaptation-assist device according to claim 47, wherein the tissue-piercing anchors are fixed to the coaptation-assist surface at or near respective ends of the two shorter sides of the sheet.

50. The leaflet coaptation-assist device according to any one of claims 29-41, wherein the tissue-piercing anchors are separate and discrete from the leaflet coaptation-assist device, and are configured to be coupled to the leaflet coaptation-assist device and the structure in situ during an implantation procedure.

51. A leaflet coaptation-assist device for promoting valve coaptation of leaflets of a mitral valve of a heart of a patient, the leaflet coaptation-assist device comprising:
a sheet having two longer sides and two shorter sides, and a lower surface that defines a coaptation-assist surface and has a convex curvature that is (a) elliptic or (b) bi-lobal having two rounded curvature shapes; and
one or more tissue anchors, which are configured to anchor the leaflet coaptation-assist device in place within the heart of the patient, such that the coaptation-assist surface is located at or below the leaflet hinge line and contacts an atrial side of the leaflets during systole, thereby enhancing leaflet coaptation, the leaflets selected from the group consisting of: native leaflets and prosthetic leaflets.

52. The leaflet coaptation-assist device according to claim 51, wherein the one or more tissue anchors are configured to anchor the leaflet coaptation-assist device in place within the heart of the patient, such that the coaptation-assist surface is located at the leaflet hinge line and contacts the atrial side of the leaflets during systole, thereby blocking motion of the leaflets into an atrium and preventing leaflet prolapse.

53. The leaflet coaptation-assist device according to claim 51, wherein the one or more tissue anchors are configured to anchor the leaflet coaptation-assist device in place within the heart of the patient, such that the coaptation-assist surface is located below the leaflet hinge line such that the leaflets partially coapt with the coaptation-assist surface during systole.

54. The leaflet coaptation-assist device according to claim 51, wherein the convex curvature is elliptic.

55. The leaflet coaptation-assist device according to claim 51, wherein the convex curvature is bi-lobal.

56. The leaflet coaptation-assist device according to claim 51, wherein the one or more tissue anchors are configured to anchor the leaflet coaptation-assist device to an annulus of the mitral valve.

57. The leaflet coaptation-assist device according to claim 51, wherein the one or more tissue anchors are configured to anchor the leaflet coaptation-assist device to an annuloplasty ring implanted on an annulus of the mitral valve.

58. The leaflet coaptation-assist device according to any one of claims 51-57, further comprising a curved stiffener that is coupled to the sheet and provides the convex curvature.

59. The leaflet coaptation-assist device according to claim 58, wherein the curved stiffener comprises a material having a shape memory.

60. The leaflet coaptation-assist device according to any one of claims 51-57, wherein the shorter sides include respective stiffened portions.

61. The leaflet coaptation-assist device according to claim 60, wherein each of the stiffened portions is formed by two layers of the sheet or by a folding of the sheet.

62. The leaflet coaptation-assist device according to claim 60, wherein the stiffened portions comprise respective stiffener elements.

63. The leaflet coaptation-assist device according to any one of claims 51-57, wherein the sheet has exactly four sides.

64. The leaflet coaptation-assist device according to claim 63, wherein the sheet has a shape of a quadrilateral.

65. The leaflet coaptation-assist device according to claim 63, wherein the sheet has two straight sides and two curved sides.

66. The leaflet coaptation-assist device according to claim 65, wherein the two shorter sides are straight and the two longer sides are curved.

67. The leaflet coaptation-assist device according to claim 65, wherein the two longer sides are straight and the shorter sides are curved.

68. The leaflet coaptation-assist device according to any one of claims 51-57, wherein the one or more tissue anchors comprise a plurality of tissue-piercing anchors, which are configured to be coupled to a structure selected from the group consisting of: an annulus of the mitral valve and an annuloplasty ring implanted on the annulus of the mitral valve.

69. The leaflet coaptation-assist device according to claim 68, wherein the tissue-piercing anchors comprise respective helical tissue-piercing coupling-elements.

70. The leaflet coaptation-assist device according to claim 68, wherein the tissue-piercing anchors are fixed to the coaptation-assist surface along the two shorter sides of the sheet.

71. The leaflet coaptation-assist device according to claim 70, wherein the tissue-piercing anchors are fixed to the coaptation-assist surface at or near respective centers of the two shorter sides of the sheet.

72. The leaflet coaptation-assist device according to claim 70, wherein the tissue-piercing anchors are fixed to the coaptation-assist surface at or near respective ends of the two shorter sides of the sheet.

73. The leaflet coaptation-assist device according to claim 68, wherein the tissue-piercing anchors are separate and discrete from the leaflet coaptation-assist device, and are configured to be coupled to the leaflet coaptation-assist device and the structure in situ during an implantation procedure.

74. A leaflet coaptation-assist device for promoting valve coaptation of leaflets of an atrioventricular valve of a heart of a patient, the leaflet coaptation-assist device comprising:
a thick structure having a lower surface that provides a coaptation-assist surface, wherein the thick structure is curved on a ventricular side thereof; and
one or more tissue anchors, which are configured to anchor the leaflet coaptation-assist device in place within the heart of the patient, such that the coaptation-assist surface is located at or below the leaflet hinge line and contacts an atrial side of the leaflets during ventricular systole, thereby enhancing leaflet coaptation, the leaflets selected from the group consisting of: native leaflets and prosthetic leaflets.

75. The leaflet coaptation-assist device according to claim 74, wherein the thick structure comprises a solid foam.

76. The leaflet coaptation-assist device according to claim 74, wherein the thick structure comprises at least one balloon.

77. The leaflet coaptation-assist device according to claim 74, wherein the atrioventricular valve is a mitral valve.

78. The leaflet coaptation-assist device according to claim 74, wherein the atrioventricular valve is a tricuspid valve.

79. The leaflet coaptation-assist device according to claim 74, wherein the one or more tissue anchors are configured to anchor the leaflet coaptation-assist device in place within the heart of the patient, such that the coaptation-assist surface is located at the leaflet hinge line and contacts the atrial side of the leaflets during ventricular systole, thereby blocking motion of the leaflets into an atrium and preventing leaflet prolapse.

80. The leaflet coaptation-assist device according to claim 74, wherein the one or more tissue anchors are configured to anchor the leaflet coaptation-assist device in place within the heart of the patient, such that the coaptation-assist surface is located below the leaflet hinge line such that the leaflets partially coapt with the coaptation-assist surface during ventricular systole.

81. The leaflet coaptation-assist device according to claim 74, wherein the one or more tissue anchors are configured to anchor the leaflet coaptation-assist device to an annulus of the atrioventricular valve.

82. The leaflet coaptation-assist device according to claim 74, wherein the one or more tissue anchors are configured to anchor the leaflet coaptation-assist device to an annuloplasty ring implanted on an annulus of the atrioventricular valve.

83. The leaflet coaptation-assist device according to claim 74, wherein the one or more tissue anchors comprise a plurality of tissue-piercing anchors, which are configured to be coupled to a structure selected from the group consisting of: an annulus of the atrioventricular valve and an annuloplasty ring implanted on the annulus of the atrioventricular valve.

84. The leaflet coaptation-assist device according to claim 83, wherein the tissue-piercing anchors comprise respective helical tissue-piercing coupling-elements.

85. The leaflet coaptation-assist device according to claim 83, wherein the coaptation-assist surface has four sides, and wherein the tissue-piercing anchors are fixed to the coaptation-assist surface along two shorter sides of the coaptation-assist surface.

86. The leaflet coaptation-assist device according to claim 85, wherein the tissue-piercing anchors are fixed to the coaptation-assist surface at or near respective centers of the two shorter sides of the coaptation-assist surface.

87. The leaflet coaptation-assist device according to claim 85, wherein the tissue-piercing anchors are fixed to the coaptation-assist surface at or near respective ends of the two shorter sides of the coaptation-assist surface.

88. The leaflet coaptation-assist device according to claim 83, wherein the tissue-piercing anchors are separate and discrete from the leaflet coaptation-assist device, and are configured to be coupled to the leaflet coaptation-assist device and the structure in situ during an implantation procedure.

89. The leaflet coaptation-assist device according to claim 74, wherein the one or more tissue anchors comprise a non-tissue-piercing anchor, which is configured to couple the leaflet coaptation-assist device to a native cardiac structure.

90. The leaflet coaptation-assist device according to claim 89, wherein the non-tissue-piercing anchor comprises a stent, which is configured to couple the leaflet coaptation-assist device to atrial tissue by radial force and friction.

91. The leaflet coaptation-assist device according to claim 89, wherein the non-tissue-piercing anchor comprises one or more ventricular loops, which are configured to couple the leaflet coaptation-assist device to a ventricular wall by radial force and friction.

92. The leaflet coaptation-assist device according to claim 91, wherein the one or more ventricular loops are configured to contact one or more of the atrioventricular leaflets at the commissural level and clip the one or more leaflets within the one or more ventricular loops to stabilize anchoring of the leaflet coaptation-assist device.

93. The leaflet coaptation-assist device according to claim 74, wherein the thick structure has a greatest thickness in the atrial-ventricular direction of at least 3 mm.

94. The leaflet coaptation-assist device according to claim 93, wherein the thick structure has a greatest thickness in the atrial-ventricular direction of no more than 40 mm.

95. The leaflet coaptation-assist device according to any one of claims 74-94, wherein the thick structure is multi-curved on the ventricular side thereof.

96. The leaflet coaptation-assist device according to any one of claims 74-94, wherein the thick structure is flat on an atrial side thereof.

97. A leaflet coaptation-assist device for promoting valve coaptation of leaflets of a tricuspid valve of a heart of a patient, the leaflet coaptation-assist device comprising:
a sheet, which is shaped so as to define three spokes extending radially outward from a central hub region, and which as a lower surface that defines a coaptation-assist surface; and
one or more tissue anchors, which are configured to anchor the leaflet coaptation-assist device in place within the heart of the patient, such that the coaptation-assist surface is located at or below the leaflet hinge line and contacts an atrial side of the leaflets during ventricular systole, thereby enhancing leaflet coaptation, the leaflets selected from the group consisting of: native leaflets and prosthetic leaflets.

98. The leaflet coaptation-assist device according to claim 97, wherein the three spokes are collectively shaped so as to define a deltoid curve.

99. The leaflet coaptation-assist device according to claim 97, wherein radially-outward ends of the spokes include respective stiffened portions.

100. The leaflet coaptation-assist device according to any one of claims 97-99, wherein the one or more tissue anchors comprise a non-tissue-piercing anchor, which comprises one or more ventricular loops, which extend in a ventricular direction from the sheet to stabilize the leaflet coaptation-assist device within a ventricular chamber.

101. The leaflet coaptation-assist device according to any one of claims 97-99, wherein the one or more tissue anchors comprise a plurality of tissue-piercing anchors.
